(19) United States
(12) Patent Application Publication
(10) Pub. No.: US 2024/0228948 A9
(43) Pub. Date: Jul. 11, 2024

(54) DYNAMIC MONOSACCHARIDE CONTROL PROCESSES

(71) Applicant: JANSSEN BIOTECH, INC., Horsham, PA (US)

(72) Inventors: James Vincent PRICE, Malvern, PA (US); Stefanie BERGES-VORSANGER, Malvern, PA (US); Amalie LEVY, Malvern, PA (US); Steven SAVAGE, Malvern, PA (US); Abbey WEITH, Malvern, PA (US)

(73) Assignee: JANSSEN BIOTECH, INC., Horsham, PA (US)

(21) Appl. No.: 17/769,373

(22) PCT Filed: Oct. 16, 2020
(86) PCT No.: PCT/US20/55925

§ 371 Date: Apr. 15, 2022

Related U.S. Application Data
(60) Provisional application No. 62/923,185, filed on Oct. 18, 2019.
(60) Provisional application No. 62/923,204, filed on Oct. 18, 2019.
(60) Provisional application No. 62/923,217, filed on Oct. 18, 2019.
(60) Provisional application No. 63/000,361, filed on Mar. 26, 2020.
(60) Provisional application No. 63/000,366, filed on Mar. 26, 2020.
(60) Provisional application No. 63/000,371, filed on Mar. 26, 2020.

(51) Int. Cl. C12N 5/00; C12M 1/00; C12M 1/06; C12M 1/34; C12M 1/36

(52) U.S. Cl. CPC C12N 5/00; C12M 27/06; C12M 29/06; C12M 41/32; C12M 41/36; C12M 41/48

(57) ABSTRACT

Materials and methods to control a nutrient feed in a cell culture process is provided. A sample is received from a bioreactor comprising a cell culture. A viable cell density and a residual nutrient measurement are determined from the received sample. A daily nutrient feeding target is calculated based on the viable cell density and the residual nutrient measurement. The nutrient is fed to the bioreactor according to the calculated daily nutrient feeding target.

### CROSS REFERENCE TO RELATED APPLICATION

The present application claims benefit of U.S. Provisional Patent Application No. 62/923,185 filed Oct. 18, 2019, U.S. Provisional Patent Application No. 62/923,204 filed Oct. 18, 2019, U.S. Provisional Patent Application No. 62/923,217 filed Oct. 18, 2019, U.S. Provisional Patent Application No. 63/000,361 filed Mar. 26, 2020, U.S. Provisional Patent Application No. 63/000,366 filed Mar. 26, 2020, and U.S. Provisional Patent Application No. 63/000,371 filed Mar. 26, 2020. The entire contents of which are hereby incorporated by reference.

### BACKGROUND

Cell culture productivity relies on optimization of culture medium management enabling high cell densities. Nutrient feeding is an important parameter for process optimization. High cell density processes can require substantial amounts of nutrients and the daily requirements vary with cell types or density.

### SUMMARY

Improved materials and methods for the prediction of daily nutrient target requirements for a given cell line is needed and is addressed by the present invention. One aspect of the disclosed technology relates to a method of controlling a nutrient feed in a cell culture process. A sample may be received from a bioreactor comprising a cell culture. A viable cell density and a residual nutrient measurement may be determined from the received sample. A daily nutrient feeding target may be calculated based on the viable cell density and the residual nutrient measurement. The nutrient may be fed to the bioreactor according to the calculated daily nutrient feeding target.

In one embodiment, a daily residual nutrient concentration may be maintained in the bioreactor within a predetermined range.

In one embodiment, the daily nutrient feeding target may be recalculated based on the viable cell density and the residual nutrient measurement on a daily basis.

In one embodiment, the nutrient may be selected from glucose, glutamate, galactose, lactate, and glutamine.

In one embodiment, the nutrient may include one or more monosaccharides.

In one embodiment, the residual nutrient measurement may comprise assaying a nutrient concentration in the bioreactor.

In one embodiment, the residual nutrient measurement may comprise performing one or more of offline nutrient measurement and inline nutrient measurement.

In one embodiment, the bioreactor may be any bioreactor known in the art. In some embodiments, the capacity of the bioreactor ranges from about 15 ml to about 15,000 L. In one embodiment, the bioreactor may be one or more of the following: a Chinese hamster ovary (CHO) cell bioreactor, and a 5 L bioreactor. Other mammalian cell types that may be used in manufacturing biologics besides CHO. Non-limiting examples of such mammalian cell types include HEK, 293 and PerC6. This process may be also used for other non-mammalian cell types, such as, for example, yeast and bacteria.

In some embodiments, cells in the bioreactor are any type of cells known in the art. In one embodiment, cells in the bioreactor may be mammalian cells. In one embodiment, cells in the bioreactor may be bacterial, yeast, or insect cells.

In one embodiment, the cells may be CHO cells, recombinant CHO cells or mixtures thereof.

In one embodiment, the daily nutrient feeding target may be calculated based at least in part on a global average consumption value and a growth profile predetermined in advance from multiple runs of the bioreactor from at least 6 cell lines.

Another aspect of the disclosed technology relates to a method of controlling a nutrient feed in a cell culture process. A sample may be received from a vessel comprising a cell culture. A viable cell density and a residual nutrient measurement may be determined from the received sample. A daily nutrient feeding target may be calculated based on the viable cell density and the residual nutrient measurement. The nutrient may be fed to the vessel according to the calculated daily nutrient feeding target.

In one embodiment, the vessel may be a flask.

In one embodiment, the nutrient may be selected from glucose, glutamate, galactose, lactate, and glutamine. In one embodiment, the nutrient may be selected from amino acids or vitamins.

A further aspect of the disclosed technology relates to a method of balancing a glucose feed in a cell growth process. A viable cell density may be periodically determined during the cell growth process. A glucose concentration may be periodically measured during the cell growth process. A glucose feeding target of a nutrient may be periodically adjusted based on the viable cell density and the glucose concentration. The glucose may be periodically fed to the cell growth process according to the glucose feeding target.

One aspect of the disclosed technology relates to a system of controlling a nutrient feed in a cell culture process. A processor may be in communication with a bioreactor and a nutrient feed system. The cell culture process may take place inside the bioreactor. The nutrient feed system may feed a nutrient to the bioreactor. The processor may determine a viable cell density and a residual nutrient measurement from a sample retrieved from the bioreactor. A daily nutrient feeding target may be calculated based on the viable cell density and the residual nutrient measurement. The processor may direct the nutrient feed system to feed the nutrient to the bioreactor according to the calculated daily nutrient feeding target.

In one embodiment, the nutrient feed system may provide a continuous or discontinuous feed of the nutrient during the cell culture process.

In one embodiment, the nutrient may be selected from glucose, glutamate, galactose, lactate, and glutamine.

In one embodiment, the nutrient may include one or more monosaccharides.

Another aspect of the disclosed technology relates to a system of balancing a glucose feed in a cell growth process. A processor may be in communication with a glucose feed system that feeds glucose during the cell growth process. The processor may periodically determine a viable cell density and a glucose concentration measured during the cell growth process. The processor may periodically adjust a glucose feeding target based on the viable cell density and the glucose concentration. The processor may periodically direct the glucose feed system to feed glucose to the cell growth process according to the glucose feeding target.

A further aspect of the disclosed technology relates to a system of preventing glycation in a cell culture process. A processor may be in communication with a bioreactor and a nutrient feed system. The cell culture process may take place inside the bioreactor. The nutrient feed system may feed a nutrient to the bioreactor. The processor may determine a residual amount of a nutrient within a sample retrieved from the bioreactor. The processor may determine a consumed amount of the nutrient since previous feeding based on the residual amount of the nutrient. The processor may determine a viable cell density within the sample. The processor may calculate a predicted consumption amount of the nutrient to be consumed before next feeding based on the consumed amount of the nutrient and the viable cell density. The processor may calculate a target amount of the nutrient for current feeding based on the predicted consumption amount of the nutrient and a predetermined residual nutrient target before next feeding. The processor may direct the nutrient feed system to feed the nutrient to the bioreactor according to the calculated target amount of the nutrient.

One aspect of the disclosed technology relates to a method of modulating an amount of glycation of an agent in a cell culture process. The cell culture process may take place inside a bioreactor. A sample from the bioreactor may be received. A residual amount of a nutrient may be measured from the received sample. A consumed amount of the nutrient since previous feeding may be determined based on the residual amount of the nutrient. A viable cell density may be determined from the received sample. A predicted consumption amount of the nutrient to be consumed before next feeding may be calculated based on the consumed amount of the nutrient and the viable cell density. A target amount of the nutrient for current feeding may be calculated based on the predicted consumption amount of the nutrient and a predetermined residual nutrient target before next feeding. The nutrient may be fed to the bioreactor according to the calculated target amount of the nutrient.

In one embodiment, a predicted viable cell density between the current feeding and the next feeding may be determined based at least in part on the determined viable cell density. A nutrient consumption rate may be determined based at least in part on the consumed amount of the nutrient. The predicted consumption rate may be calculated based on the predicted viable cell density and the nutrient consumption rate;

In one embodiment, the feeding may take place on a daily basis.

Another aspect of the disclosed technology relates to a method of controlling a glucose feed in a cell culture process. A sample is received from a bioreactor comprising a cell culture. A residual amount of glucose is measured from the received sample. A sample time is determined when the sample is received from the bioreactor. A processor compares the residual amount of glucose with a predetermined glucose target. The processor calculates a consumed amount of glucose. When the residual amount of glucose is greater than the predetermined glucose target, the processor determines the consumed amount of glucose by determining an amount of glucose that is consumed between a previous day and a present day during the cell culture process. When the residual amount of glucose is not greater than the predetermined glucose target, the processor determines the consumed amount of glucose based on a difference between the predetermined glucose target and the residual amount of glucose. The processor calculates an integrated viable cell density. The processor calculates a predetermined viable cell density for a following day based on the integrated viable cell density. The processor calculates a specific glucose consumption rate based on the consumed amount of glucose and the integrated viable cell density. The processor calculates a predicted glucose consumption amount by multiplying the specific glucose consumption rate by the predetermined viable cell density for the following day. The processor calculates a glucose target by summing the predetermined glucose consumption amount and a predetermined glucose minimum amount. Glucose is fed to the bioreactor according to the glucose target.

In one embodiment, the feeding may take place on a daily basis.

Further features of the present disclosure, and the advantages offered thereby, are explained in greater detail hereinafter with reference to specific embodiments illustrated in the accompanying drawings, wherein like elements are indicated by like reference designators.

### BRIEF DESCRIPTION OF THE DRAWINGS

Reference will now be made to the accompanying drawings, which are not necessarily drawn to scale, and which are incorporated into and constitute a portion of this disclosure, illustrate various implementations and aspects of the disclosed technology and, together with the description, explain the principles of the disclosed technology. In the drawings:

FIG. 1 is a schematic diagram of an example environment that may be used to implement one or more embodiments of the present disclosure.

FIG. 2 is a schematic diagram of an example environment that may be used to implement one or more embodiments of the present disclosure.

FIG. 3 is a flow chart of glucose algorithm according to one aspect of the disclosed technology.

FIG. 4 is an example of an automated process for feeding glucose according to one aspect of the disclosed technology.

FIG. 5 is a block diagram of a nutrient feed control system according to one aspect of the disclosed technology.

FIG. 6A is an example table illustrating experimentally determined residual glucose targets by culture day according to one aspect of the disclosed technology.

FIG. 6B is another example table illustrating experimentally determined residual glucose targets by culture day according to one aspect of the disclosed technology.

FIG. 6C is an example table illustrating count of cell lines and bioreactor runs used to calculate expected cell growth behavior according to one aspect of the disclosed technology.

FIG. 7 is a chart illustrating median and IQR for fold changes in ΔIVCD over time according to one aspect of the disclosed technology.

FIG. 8 is a chart illustrating median and IQR for ΔIVCD/VCD over time according to one aspect of the disclosed technology.

FIGS. 9A-B illustrate charts of percent error in VCD prediction over time according to one aspect of the disclosed technology.

FIGS. 10A-C illustrate charts of measured residual glucose levels with different cell lines over time according to one aspect of the disclosed technology.

FIGS. 11A-B illustrate additional charts of measured residual glucose levels with different cell lines over time according to one aspect of the disclosed technology.

FIG. 12 illustrates a chart of measured residual glucose levels with RSV over time according to one aspect of the disclosed technology.

FIG. 13 illustrates a chart of measured residual glucose levels with cell line CHO6 over time according to one aspect of the disclosed technology.

FIG. 14 illustrates median expected and median expected

$$\frac{Δ {IVCD}_{d + 1}}{Δ {IVCD}_{d}}$$

and median expected

$$\frac{Δ {IVCD}_{d + 1}}{{VCD}_{d + 1}}$$

value calculated from database of Janssen cell line.

FIG. 15 is a flow chart of a process performed by a nutrient feed control system according to one aspect of the disclosed technology.

FIG. 16 is an example illustration of data entry associated with a bioreactor according to one aspect of the disclosed technology.

FIG. 17 is an example illustration of data entry associated with bioreactors according to one aspect of the disclosed technology.

FIG. 18 is another example illustration of data entry associated with a bioreactor according to one aspect of the disclosed technology.

FIG. 19 is yet another example illustration of data entry associated with a bioreactor according to one aspect of the disclosed technology.

FIG. 20 is an example illustration of selecting various data import files according to one aspect of the disclosed technology.

FIG. 21 is an additional example illustration of data entry associated with a bioreactor according to one aspect of the disclosed technology.

FIG. 22 is another example illustration of data entry associated with a bioreactor according to one aspect of the disclosed technology.

FIG. 23 is yet another example illustration of data entry associated with a bioreactor according to one aspect of the disclosed technology.

FIG. 24 is an example illustration of a user interface in connection with data entry associated with a bioreactor according to one aspect of the disclosed technology.

FIG. 25 is another example illustration of a user interface in connection with data entry associated with a bioreactor according to one aspect of the disclosed technology.

FIG. 26 is yet another example illustration of a user interface in connection with data entry associated with a bioreactor according to one aspect of the disclosed technology.

FIG. 27 is an example illustration of a user interface in connection with data entry associated with a bioreactor according to one aspect of the disclosed technology.

FIG. 28 is an example illustration of data entry associated with a bioreactor according to one aspect of the disclosed technology.

FIG. 29 is another example illustration of data entry associated with a bioreactor according to one aspect of the disclosed technology.

FIG. 30 is yet another example illustration of data entry associated with a bioreactor according to one aspect of the disclosed technology.

FIG. 31 is an example illustration of data entry associated with a bioreactor according to one aspect of the disclosed technology.

FIG. 32 is another example illustration of data entry associated with a bioreactor according to one aspect of the disclosed technology.

FIG. 33 is yet another example illustration of data entry associated with a bioreactor according to one aspect of the disclosed technology.

FIG. 34 is an example illustration of selecting various data import files according to one aspect of the disclosed technology.

FIG. 35 is an example illustration of data entry associated with a bioreactor according to one aspect of the disclosed technology.

FIG. 36 is another example illustration of data entry associated with a bioreactor according to one aspect of the disclosed technology.

FIG. 37 is an example flow chart illustrating a process of controlling a nutrient feed in a cell culture process.

FIG. 38 is another example flow chart illustrating a process of controlling a nutrient feed in a cell culture process.

FIG. 39 is an example flow chart illustrating a process of balancing a glucose feed in a cell growth process.

FIG. 40 is an example flow chart illustrating a process of controlling a glucose feed in a cell culture process.

FIG. 41 is an example flow chart illustrating a process of modulating an amount of glycation of an agent in a cell culture process.

FIG. 42 is an example flow chart illustrating a process of controlling a glucose feed in a cell culture process.

FIG. 43 is another example flow chart illustrating a process of controlling a glucose feed in a cell culture process.

### DETAILED DESCRIPTION

Some implementations of the disclosed technology will be described more fully with reference to the accompanying drawings. This disclosed technology may, however, be embodied in many different forms and should not be construed as limited to the implementations set forth herein. The components described hereinafter as making up various elements of the disclosed technology are intended to be illustrative and not restrictive. Many suitable components that would perform the same or similar functions as components described herein are intended to be embraced within the scope of the disclosed electronic devices and methods. Such other components not described herein may include, but are not limited to, for example, components developed after development of the disclosed technology.

It is also to be understood that the mention of one or more method steps does not preclude the presence of additional method steps or intervening method steps between those steps expressly identified.

It is to be noted, unless otherwise clear from the context, that the term “a” or “an” entity refers to one or more of that entity; for example, “an amino acid,” is understood to represent one or more proteins. As such, the terms “a” (or “an”), “one or more,” and “at least one” can be used interchangeably herein.

The term “nutrient” may refer to any compound, molecule, or substance used by an organism to live, grow, or otherwise add biomass. Examples of nutrients may include carbohydrate sources (e.g., simple sugars such as glucose, galactose, maltose or fructose, or more complex sugars), amino acids, vitamins (e.g., B group vitamins (e.g., B12), vitamin A vitamin E, riboflavin, thiamine and biotin). In the present invention, one or more nutrients may be utilized as a surrogate molecule to determine the amount of total nutrient media to add to a bioreactor. In some embodiments, the term “nutrient” may refer to simple sugars, vitamins, and amino acids.

The term “amino acid” may refer any of the twenty standard amino acids, i.e., glycine, alanine, valine, leucine, isoleucine, methionine, proline, phenylalanine, tryptophan, serine, threonine, asparagine, glutamine, tyrosine, cysteine, lysine, arginine, histidine, aspartic acid and glutamic acid, single stereoisomers thereof, and racemic mixtures thereof. The term “amino acid” can also refer to the known non-standard amino acids, e.g., 4-hydroxyproline, hydroxy-proline, s-sulfocysteine, phosphotyrosine, ε-N,N,N-trimethyllysine, 3-methylhistidine, 5-hydroxylysine, O-phosphoserine, γ-carboxyglutamate, ε-N-acetyllysine, ω-N-methylarginine, N-acetylserine, N,N,N-trimethylalanine, N-formylmethionine, γ-aminobutyric acid, histamine, dopamine, thyroxine, citrulline, ornithine, β-cyanoalanine, homocysteine, azaserine, and S-adenosylmethionine. In some embodiments, the amino acid is glutamate, glutamine, lysine, tyrosine or valine. In some embodiments, the amino acid is glutamate or glutamine.

The terms “nutrient media,” “feed media,” “feed,” “total feed,” and “total nutrient media” may be used interchangeably, and may include a “complete” media used to grow, propagate, and add biomass to a cell line. Nutrient media may be distinguished from a substance or simple media which by itself is not sufficient to grow and propagate a cell line. Thus, for example, glucose or simple sugars by themselves are not nutrient media, since in the absence of other required nutrients, they would not be sufficient to grow and propagate a cell line.

In one aspect, the present invention teaches a carbohydrate control algorithm used to balance glucose feeds in bioreactors. such as CHO cell bioreactors. The disclosed system has advantages over the art in that it leverages data that is collected (automatically or not) to adjust glucose in the bioreactors thereby enhancing glycation prevention, reduces bioreactor crashing due to glucose underfeeding, dynamically measures cell response to glucose and adjusts glucose to any organism (CHO is exemplified). It is semi-automated or fully automated and organism agnostic, so other mammalian cells could be used (e.g., CAR-Ts, CHOs, etc.). The system is dynamic in that, inter alia, it learns periodically from for example glucose measures within the system. The external (to the bioreactor) system contains the algorithm, which directs a glucose feed system (standard, off the shelf kit is suitable for use with this system). Off the shelf glucose feed kits are configurable in many ways (multi feeds possible, various locations, continuous or discontinuous feeds possible). Continuous systems could be used, such as pre-programmed non-feedback controlled continuous feeding of cell cultures (the system would simply split the glucose bolus up, over time). This can be automated to provide the glucose data to the algorithm automatically. Adjustments to the glucose feed are based on the data, using a glucose analyzer. In some embodiments, the disclosed system is used to produce commodity chemicals other than glycoproteins, using mammalian cells, yeast, or bacteria.

In one embodiment, the disclosed system includes an automated process with feedback control during the cell culture process.

In one embodiment, both lactate and glucose are measured, and step changes (0.5 g/L at a time) are made to the glucose target.

In one embodiment, only glucose is measured. By using only glucose that streamlines the process in the lab and makes it easier for the bioreactor operators to use the algorithm and feed glucose appropriately.

In one embodiment, the algorithm only uses cell density measurements and has pre-programmed global glucose consumption values.

In one embodiment, glucose target is calculated once per day. The algorithm may be updated to allow for multiple measurements and the target being for the next 24 hrs.

The algorithm concept originated from taking the thought process human operators used and translating that to a flow diagram that used measured glucose and lactate to make step changes to increase or decrease the glucose target.

The disclosed glucose algorithm tends to be more accurate than a human operator because the human operator tends to underestimate glucose (leading to depletion events) or overestimate glucose targets to ensure that glucose is not depleted. The disclosed algorithm is better at controlling glucose at a desired level than a human operator likely could. The algorithm has a desired glucose to maintain level, e.g., which may be set to a value between 1 and 2 g/L. The disclosed method may be used for other monosaccharides, nutrients, etc. Non-limiting examples of such monosaccharides or nutrients include glutamate, galactose, lactate, and glutamine.

The disclosed technology may minimize glucose to avoid glycation.

In some embodiments, the methods disclosed herein may increase the quantity of a bioproduct produced, or decrease bioproduct production time, in a bioreactor cell culture producing the bioproduct. The disclosed method may include (a) intermittently or continuously analyzing the concentration of one or more nutrient in the bioreactor cell culture; and (b) adding to the bioreactor cell culture additional nutrient media when the concentration of the one or more nutrients is lower than a target value.

In some embodiments, additional nutrient media may be added to the bioreactor cell culture in an amount sufficient to maintain a substantially stable concentration of the amino acid throughout a bioreactor process.

In some embodiments, the bioreactor cell culture may include Chinese Hamster Ovary (CHO) cells, HEK-293 cells, or VERO cells. In some embodiments, the bioproduct may be an antibody or antibody-like polypeptide.

In one embodiment, the methods of the present invention may be performed in the presence of any cell culture media. For example, the bioreactor process may be performed in the presence of serum-free media, protein-free media (including, but not limited to, protein-free media containing protein hydrolysates), or chemically defined media.

Various analytical devices may be used in the present invention. The analytical devices may include any instrument or process that can detect and/or quantify a surrogate molecule or marker, e.g., an amino acid or other substituents of cell culture media (e.g., a vitamin, a mineral, an ion, sugar, etc.). The analytical device may be an apparatus for performing gas chromatography, HPLC, cation exchange chromatography, anion exchange chromatography, size exclusion chromatography, an enzyme-catalyzed assay, and/or a chemical reaction assay.

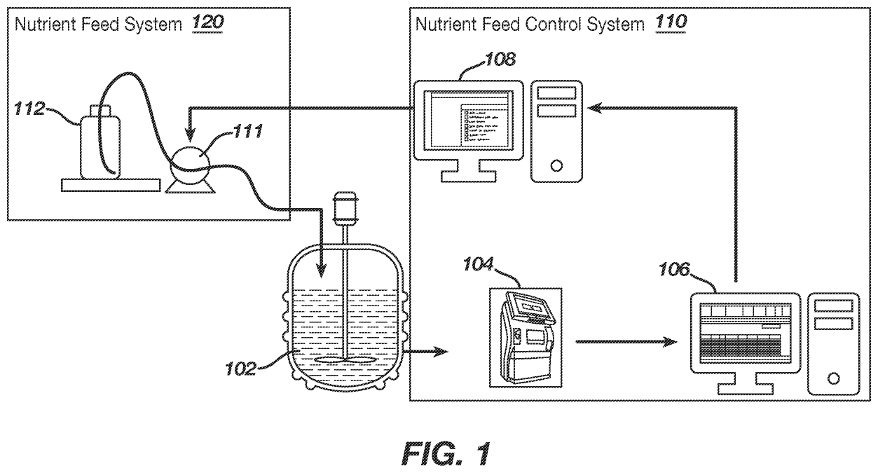

As shown in FIG. 1, a production reactor 102 may include mammalian cell culture. The production reactor 102 may be a bioreactor, a cell culture reactor or a sample bioreactor. The production reactor 102 may be at least one of the following: a well plate, a shake flask, a bench top vessel, and a commercial scale (e.g., 15 kL) stainless steel reactor. Reaction sample may be withdrawn from the production reactor 102, and sent to a nutrient feed control system 110. The nutrient feed control system 110 may include a glucose measurement system 104 that performs glucose measurement. The glucose measurement may be performed either offline or online. The nutrient feed control system 110 may also include a glucose target prediction system 106 that receives glucose measurement from the glucose measurement system 104, and performs glucose target prediction. The nutrient feed control system 110 may include a glucose calculation system 108 may then use the predicted glucose target to calculate an amount of glucose to add, and send an instruction to a nutrient feed system 120. In one embodiment, processes performed by the glucose measurement system 104, the glucose target prediction system 106 and the glucose calculation system 108 may be completed by one or more processors.

The nutrient feed system 120 may include a pump 111 which feeds the correct amount of glucose from glucose feed 112 to the production reactor 102.

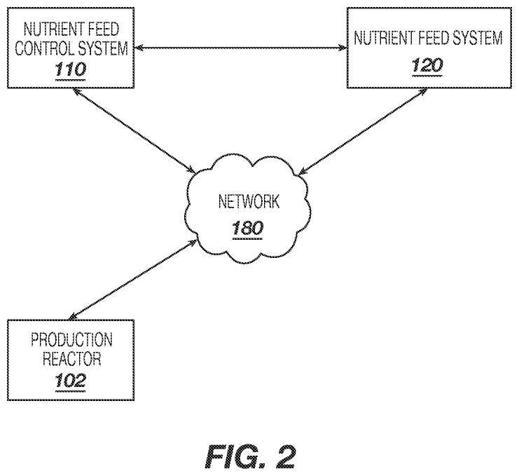

FIG. 2 illustrates schematic diagram of an example environment that may be used to implement one or more embodiments of the present disclosure. The nutrient feed control system 110 may communicate with the production reactor 102 and the nutrient feed system 120 over a network 180. The nutrient feed control system 110 may direct the nutrient feed system 120 to feed one or more nutrients to the production reactor 102.

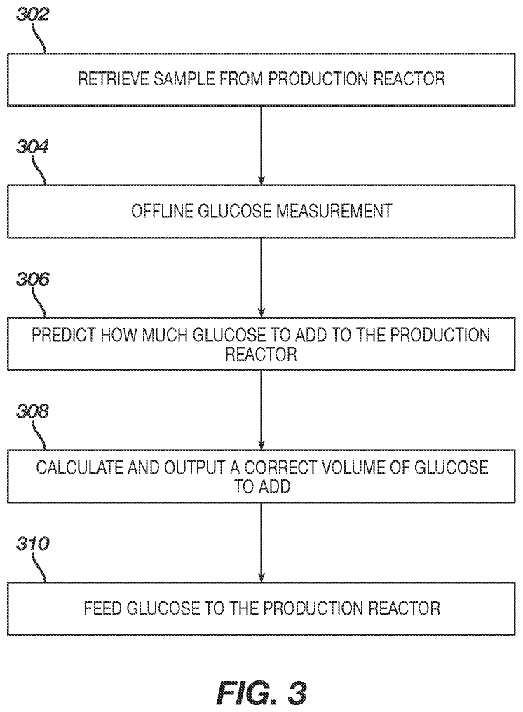

FIG. 3 illustrates a flow diagram for glucose algorithm. At 302, the production reactor 102 may provide sample. At 304, the glucose measurement system 104 may receive the sample, and conduct glucose measurement. At 306, the glucose target prediction system 106 may predict how much glucose to add to the production reactor 102. At 308, the glucose calculation system 108 may calculate and output a correct volume of glucose to add. At 310, the correct volume of glucose may be fed to the production reactor 102. This algorithm may be applicable to preculture. For example, the algorithm may be used to feed glucose to intensified seed trains. The algorithm may also be applicable to N−1 perfusion process and production perfusion processes.

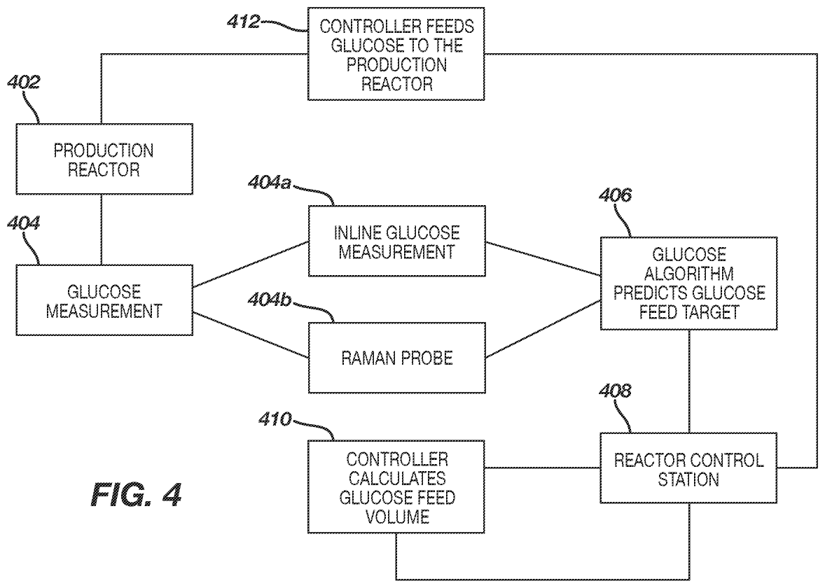

FIG. 4 illustrate an example of an automated process. At 402, the production reactor 102 may provide sample. At 404, glucose measurement may be conducted, such as inline glucose measurement (e.g., NovaFlex) at 404a, or Raman probe at 404b. An instrument may be used to measure offline pH as well as glucose and lactate. At 406, the nutrient feed control system 110 may perform predict glucose feed target. At 408, a reactor control station may process the predicted glucose feed target. At 410, a controller may calculate glucose feed volume. At 412, the controller may feed glucose to the production reactor 102.

The methods disclosed herein may increase production in subsequent bioreactor cell cultures. In some embodiments, the method may enhance the quantity of an antibody (or other bioproduct) produced, or decreasing antibody (or other bioproduct) production time, in a bioreactor cell culture producing the antibody (or other bioproduct). The method may include analyzing a culture sample (with or without extracting a sample from the bioreactor) by means of an automated sampling device (such as, for example, by means of off-line, on-line, in-line or at-line sample analysis). The method may include analyzing a culture sample (e.g., the concentration of residual glucose) by means of an automated analytical device to generate data representative of the quantity of a nutrient (or other surrogate marker). The method may include processing the generated data (e.g., from assaying residual glucose from the sample) by means of an algorithm or computer-based processing program wherein the processed data is used to determine an amount of additional nutrient media to add to the bioreactor. The method may include adding the determined amount of nutrient media determined to the bioreactor by means of an automated feed device. The method may include recording the time and amount of each nutrient media addition.

Mammalian cells may include any mammalian cells that are capable of growing in culture. Exemplary mammalian cells include, e.g., CHO cells (including CHO-K1, CHOK1SV®, CHO DUKX-B11, CHO DG44), VERO, BHK, HeLa, CV1 (including Cos; Cos-7), MDCK, 293, 3T3, C127, myeloma cell lines (especially murine), PC12, HEK-293 cells (including HEK-293T and HEK-293E), PER C6, Sp2/0, NS0 and W138 cells. Mammalian cells derived from any of the foregoing cells may also be used. In some embodiments, the bioreactor cell culture may comprise Chinese Hamster Ovary (CHO) cells, HEK-293 cells, or VERO cells.

The steps of the disclosed method may be repeated, and may occur at various intervals. In some embodiments, steps disclosed herein may be repeated greater than 10 times throughout a bioreactor process, or 10 to 1000 times, 20 to 500 times or 30 to 100 times throughout a bioreactor process. In some embodiments, steps may be repeated about every 4 minutes, 10 minutes, 30 minutes, 60 minutes, 2 hours, 3 hours, 6 hours, 8 hours, 12 hour, 16 hours, 18 hours, or 24 hours throughout a bioreactor process, or about every 4 to 18 hours, or about every 10 minutes to about every 6 hours throughout a bioreactor process. In specific embodiments, the method comprises measuring the amount of residual nutrient (e.g., residual glucose) once per day, with a target concentration of the nutrient (glucose) generated for a period about one day, or about 24 hours later. In certain embodiments, the method comprises measuring the amount of residual nutrient (e.g., residual glucose) multiple times per day, e.g., twice, three times, or four times per day, with a target concentration of the nutrient (glucose) generated for a period about 24 hours the measurement.

The steps of the methods disclosed herein may occur in a relatively short amount of time, i.e., the sampling, analysis, and addition of additional nutrient media can occur relatively quickly. In some embodiments, steps of the disclosed method are performed within about 1 minute to about 2 hours.

In some embodiments, steps of the disclosed method are performed by one or more automated devices. The terms “automatic”, “automatically”, or “automated” describe one or more mechanical devices that perform one or more tasks without any human intervention or action, except for any human intervention or action necessary to initially prepare the device or devices for task performance; or as may be required to maintain automatic operation of the device or devices. A “mechanical device” that performs one or more tasks automatically may, optionally, include a computer and the necessary instructions (code) therein to process collected data which may be used therein for decision making purposes to control and direct performance of the device or devices, such as in controlling the timing, duration, frequency, kind, and/or character of tasks to be performed.

In various embodiments, “off-line” analysis refers to permanently removing a sample from the production process and analyzing the sample at a later point in time such that the data analysis does not convey real-time or near real-time information about in-process conditions. In some embodiments, one or more analytical devices are used off-line.

In one embodiment, an analytical device (or a sensor-portion connected thereto) may be introduced directly into a bioreactor or purification unit, or the device or sensor-portion may be separated from the bioreactor or purification unit by an appropriate barrier or membrane.

In some embodiments, the analytical device may be a kit, e.g., a test strip, which can be placed in contact with the sample to give rapid determination of the cellular concentration. In some embodiment, the kit may comprise a substrate which produces a chemical and/or enzyme-linked reaction to produce a detectable signal in the presence of a surrogate marker, or a specific concentration of a surrogate marker. The detectable signal may include, e.g., a colormetric change or other visual signal. In some embodiments, the analytical device may be a disposable analytical device, e.g., a disposable test strip. Such kits may be useful do to their ease of operation and their reduced costs relative to other larger, more complicated analytical devices. Such kits may also be useful during small scale cell culture propagation to determine that optimal health and productivity of the culture.

A “traditional manufacturing process” may include (a) adding nutrient media in bolus feeds to the bioreactor at designated time points, or (b) adding glucose (or another single nutrient) to the bioreactor as the glucose (or other single nutrient) is consumed. The traditional manufacturing processes may lead to lower bioproduct yields and/or less efficient bioproduct production. In one embodiment, the disclosed system may utilize a feedback control method, wherein the concentration of one or more nutrients is monitored, and based on the concentration of that nutrient, an appropriate amount of total media is added to the bioreactor. The monitoring can be done automatically and frequently, resulting in greatly increased yields of bioproduct.

In some embodiments, the quantity of the bioproduct produced may increase significantly relative to traditional manufacturing processes. In some embodiments, the quantity of bioproduct produced may be 10% to 100% greater than the quantity of bioproduct produced by a traditional manufacturing process. In some embodiments, the quantity of bioproduct produced by the method of the present invention may be 10% 15%, 20%, 25%, 30%, 35%, 40%, 45%, 50%, 55%, 60%, 65% 70%, 80%, or 90% 100% greater than the quantity of bioproduct produced by the traditional manufacturing process.

In some embodiments, the extent of glycation of the bioproduct is reduced by at least 10%, at least 15%, at least 20%, at least 25%, at least 30%, at least 35%, at least 40% or by at least 45% in comparison to the extent of glycation of a bioproduct produced by a traditional manufacturing process.

In various embodiments, the glucose algorithms and methods described herein may be effective to achieve a residual glucose level at one day post-feeding of between 0 to 3 g/L, 0.5 to 2 g/L, 2 to 5 g/L, less than 1 g/L, or less than 2 g/L.

Various bioproducts may be envisioned in the present invention. In some embodiments, the bioproduct may be an antibody, recombinant protein, glycoprotein, or fusion protein. In some embodiments, the bioproduct may be a soluble protein. In some embodiments, the bioproduct may be an antibody, antibody fragment or modified antibody (e.g., a multivalent antibody, a domain-deleted antibody, a multimeric antibody, a hinge-modified antibody, a stabilized antibody, a multispecific antibody, a linear antibody, an scFv, a linked ScFv antibody, a multivalent linear antibody, a multivalent antibody without Fc, a Fab, a multivalent Fab, etc.).

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

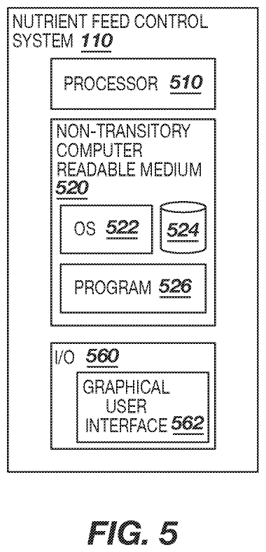

FIG. 5 is a block diagram of the nutrient feed control system 110 according to one aspect of the disclosed technology. The nutrient feed control system 110 may include one or more processors 510. The processes performed by the glucose measurement system 104, the glucose target prediction system 106 and the glucose calculation system 108 may be completed by one or more processors 510.

Referring to FIG. 5, a processor 510 may include one or more of a microprocessor, microcontroller, digital signal processor, co-processor or the like or combinations thereof capable of executing stored instructions and operating upon stored data. The processor 510 may be one or more known processing devices, such as a microprocessor from the Pentium™ family manufactured by Intel™ or the Turion™ family manufactured by AMD™. The processor 510 may constitute a single core or multiple core processor that executes parallel processes simultaneously. For example, the processor 510 may be a single core processor that is configured with virtual processing technologies. In certain embodiments, the processor 510 may use logical processors to simultaneously execute and control multiple processes. The processor 510 may implement virtual machine technologies, or other similar known technologies to provide the ability to execute, control, run, manipulate, store, etc. multiple software processes, applications, programs, etc. One of ordinary skill in the art would understand that other types of processor arrangements could be implemented that provide for the capabilities disclosed herein.

A non-transitory computer readable medium 520 may include, in some implementations, one or more suitable types of memory (e.g., such as volatile or non-volatile memory, random access memory (RAM), read only memory (ROM), programmable read-only memory (PROM), erasable programmable read-only memory (EPROM), electrically erasable programmable read-only memory (EEPROM), magnetic disks, optical disks, floppy disks, hard disks, removable cartridges, flash memory, a redundant array of independent disks (RAID), and the like), for storing files including an operating system 522, application programs (including, for example, a web browser application, a widget or gadget engine, and or other applications, as necessary), executable instructions and data. In one embodiment, the processing techniques described herein are implemented as a combination of executable instructions and data within the non-transitory computer readable medium 520. The non-transitory computer readable medium 520 may include one or more memory devices that store data and instructions used to perform one or more features of the disclosed embodiments. The non-transitory computer readable medium 520 may also include any combination of one or more databases controlled by memory controller devices (e.g., server(s), etc.) or software, such as document management systems, Microsoft™ SQL databases, SharePoint™ databases, Oracle™ databases, Sybase™ databases, or other relational or non-relational databases. The non-transitory computer readable medium 520 may include software components that, when executed by the processor 510, perform one or more processes consistent with the disclosed embodiments. In some embodiments, the non-transitory computer readable medium 520 may include a database 524 to perform one or more of the processes and functionalities associated with the disclosed embodiments. The non-transitory computer readable medium 520 may include one or more programs 526 to perform one or more functions of the disclosed embodiments. Moreover, the processor 510 may execute one or more programs 526 located remotely from the system 110. For example, the system 110 may access one or more remote programs 526, that, when executed, perform functions related to disclosed embodiments.

The system 110 may also include one or more I/O devices 560 that may comprise one or more interfaces for receiving signals or input from devices and providing signals or output to one or more devices that allow data to be received and/or transmitted by the system 110. For example, the system 110 may include interface components, which may provide interfaces to one or more input devices, such as one or more keyboards, mouse devices, touch screens, track pads, trackballs, scroll wheels, digital cameras, microphones, sensors, and the like, that enable the system 110 to receive data from one or more users. The system 110 may include a display, a screen, a touchpad, or the like for displaying images, videos, data, or other information. The I/O devices 560 may include the graphical user interface 562.

In exemplary embodiments of the disclosed technology, the system 110 may include any number of hardware and/or software applications that are executed to facilitate any of the operations. The one or more I/O interfaces 560 may be utilized to receive or collect data and/or user instructions from a wide variety of input devices. Received data may be processed by one or more computer processors as desired in various implementations of the disclosed technology and/or stored in one or more memory devices.

The networks 180 may include a network of interconnected computing devices more commonly referred to as the internet. The network 180 may be of any suitable type, including individual connections via the internet such as cellular or WiFi networks. In some embodiments, the network 180 may connect terminals, services, and mobile devices using direct connections such as radio-frequency identification (RFID), near-field communication (NFC), Bluetooth™, low-energy Bluetooth™ (BLE), WiFi™, ZigBee™, ambient backscatter communications (ABC) protocols, USB, WAN, or LAN. Because the information transmitted may be personal or confidential, security concerns may dictate one or more of these types of connections be encrypted or otherwise secured. In some embodiments, however, the information being transmitted may be less personal, and therefore the network connections may be selected for convenience over security. The network 180 may comprise any type of computer networking arrangement used to exchange data. For example, the network 180 may be the Internet, a private data network, virtual private network using a public network, and/or other suitable connection(s) that enables components in system environment to send and receive information between the components of system 100. The network 180 may also include a public switched telephone network (“PSTN”) and/or a wireless network. The network 180 may also include local network that comprises any type of computer networking arrangement used to exchange data in a localized area, such as WiFi, Bluetooth™ Ethernet, and other suitable network connections that enable components of system environment to interact with one another.

### Example 1

Glucose feeding is an important parameter for bioreactor process optimization. Moreover, high cell density processes can require substantial amounts of glucose (in the range of 5-10 g/L per day), and the daily requirement varies with cell density. Therefore, a variable glucose algorithm may be developed to predict daily glucose target requirements. The variable glucose algorithm uses viable cell density (VCD) and residual glucose measurements to calculate a daily glucose target. Implementation of the glucose algorithm may be able to achieve residual glucose levels, measured one-day post-feeding, between 0-3 g/L in six cell lines, with most residual glucose levels between 0.5-2 g/L.

A glucose feeding target for the current day (d) may be calculated from the sum of the residual glucose target for the next day (d+1) and the predicted glucose consumption between days d and d+1 (Equation 1).

Glucose Target_{d}=Predicted Glucose Consumption_{d+1}+Residual Glucose Target_{d+1} [1]

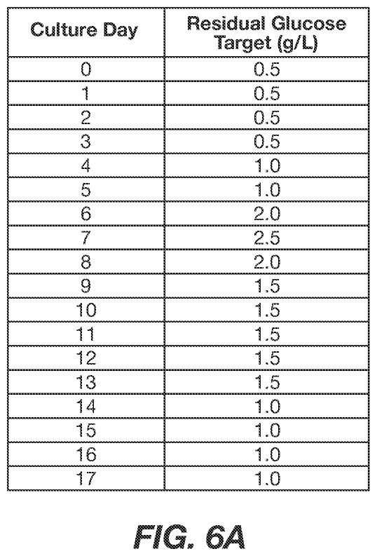

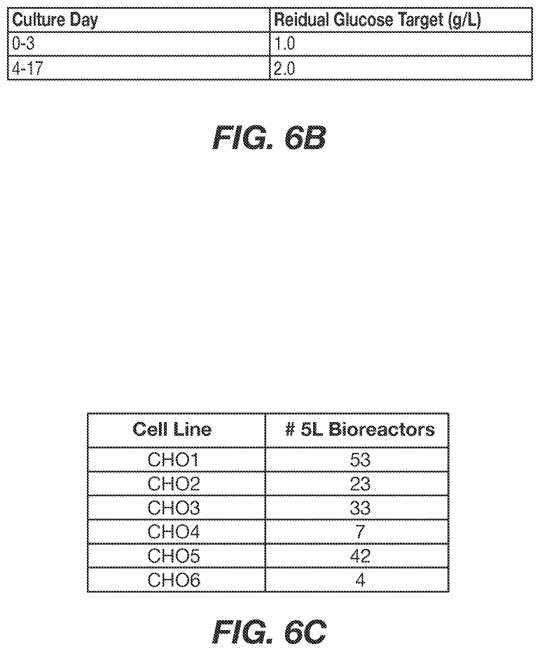

FIGS. 6A-B illustrate two tables each showing experimentally determined residual glucose targets by culture day for glucose algorithm. Residual glucose targets may be experimentally determined, and they vary between 0.5-1.5 g/L with culture day.

The predicted glucose consumption may be defined as the predicted VCD for the next culture day (VCD_{d+1}) multiplied by the specific glucose consumption rate for the current day (d) (Equation 2).

Predicted Glucose Consumption_{d+1}=Specific Glucose Consumption Rate_{d}*Predicted VCD_{d+1} [2]

In this case, the specific glucose consumption rate may be defined as the amount of glucose consumed per cell per day between days d−1 and d. The concentration of glucose consumed may be calculated as the difference between the glucose target at d−1 and the measured glucose concentration at d. Then, the concentration of glucose consumed may be normalized by the by the change in the cumulative cell density from d−1 to d(ΔIVCD_{d}) multiplied by the elapsed time between d−1 and d (Equation 3).

$$Specific Glucose Consumption {Rate}_{d} = \frac{Glucose {Target}_{d - 1} - Measured {Glucose}_{d}}{Δ {IVCD}_{d} * {time}_{d - ( d - 1 )}} \qquad [ 3 ]$$

IVCD may be approximated using the logarithmic average method in most cases. The simple average method may be used when the VCD remained unchanged from d−1 to d.

Predicted VCD for d+1 (VCD_{d+1}) may be defined as the change in IVCD from d−1 to d (ΔIVCD_{d}) multiplied by the median expected fold change in ΔIVCD from d to

$$d + 1 ( \frac{Δ {IVCD}_{d + 1}}{Δ {IVCD}_{d}} )$$

and the inverse of the median expected fold difference between ΔIVCD_{d+1} and VCD_{d+1} on

$$d + 1 ( \frac{Δ {IVCD}_{d + 1}}{{VCD}_{d + 1}} )$$

### (Equation 4).

$$Predicted {VCD}_{d + 1} = Δ {IVCD}_{d} * \frac{Median Expected \frac{Δ {IVCD}_{d + 1}}{Δ {IVCD}_{d}}}{Median Expected \frac{Δ {IVCD}_{d + 1}}{{VCD}_{d + 1}}} \qquad [ 4 ]$$

ΔIVCD_{d} is used in lieu of VCD_{d} as a measure of the viable cell density because the experimental error associated with cell-counting instruments can lead to gross over and underestimations of individual VCD measurements. Individual measurement errors have a reduced impact on IVCD, and in turn on the glucose target prediction.

Parameters to characterize the growth of cell lines in High Titer media and feeds, namely median expected fold change in ΔIVCD and ΔIVCD/VCD on day d+1, may be estimated from a database of 162 5 L bioreactor runs. Six CHOK1SV® (Lonza Sales AG) cell lines, overexpressing different proteins, and having varied phenotypes may be included, and named for brevity CHO1, CHO2, CHO3, CHO4, CHO5, and CHO6. FIG. 6C illustrates a table showing count of cell lines and bioreactor runs used to calculate expected cell growth behavior. Note that the target seeding density of these reactors may be 0.5 million viable cells per mL.

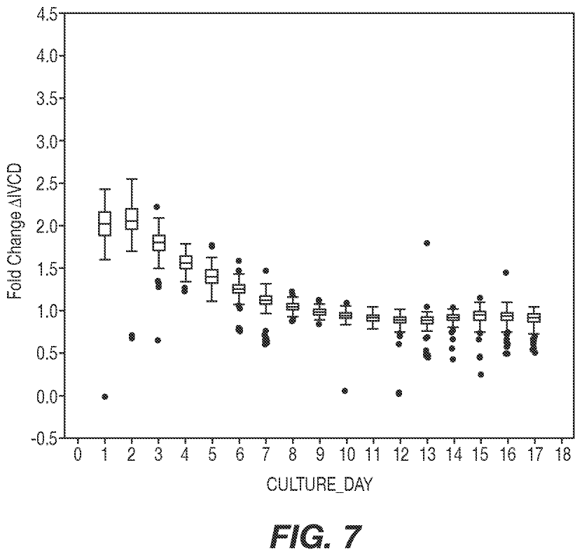

Fold change in ΔIVCD from d to d+1, may be calculated from all runs in the database as a function of culture day, irrespective of cell line or feed used. FIG. 7 illustrates calculated mean and IQR for the fold change in ΔIVCD from day d to d+1 by culture day (d) for 162 5 L bioreactor runs from six cell line with high titer media and feeds. Then, the median and IQR of the data may be computed to determine the median expected

$$\frac{Δ {IVCD}_{d + 1}}{Δ {IVCD}_{d}} .$$

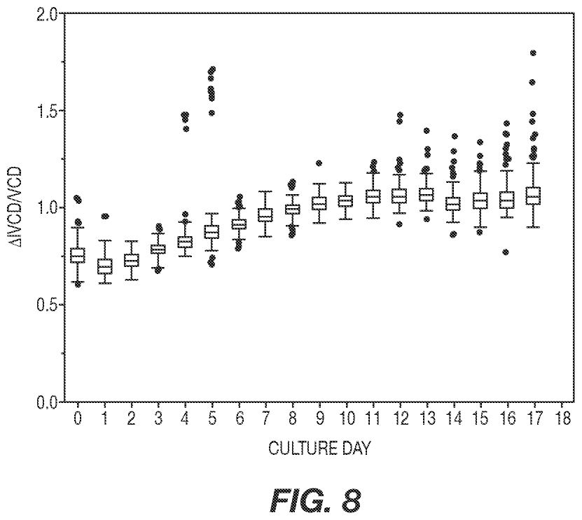

The fold difference between ΔIVCD and VCD on d+1 may be calculated from all runs in the database as a function of culture day, irrespective of cell line or feed used. FIG. 8 illustrates calculated mean and IQR for ΔIVCD/VCD on day d+1 by culture day (d) for 162 5 L bioreactor runs from six cell line with high titer media and fees. Then, the median and IQR of the data may be computed to determine the median expected

$$\frac{Δ {IVCD}_{d + 1}}{{VCD}_{d + 1}} .$$

The error in VCD prediction may be calculated as the difference between the predicted and measured VCD for day d+1 (Equation 5).

$$\% Error {VCD}_{d + 1} = \frac{( Predicted {VCD}_{d + 1} - Measured {VCD}_{d + 1} )}{Measured {VCD}_{d + 1}} * 100 \% \qquad [ 5 ]$$

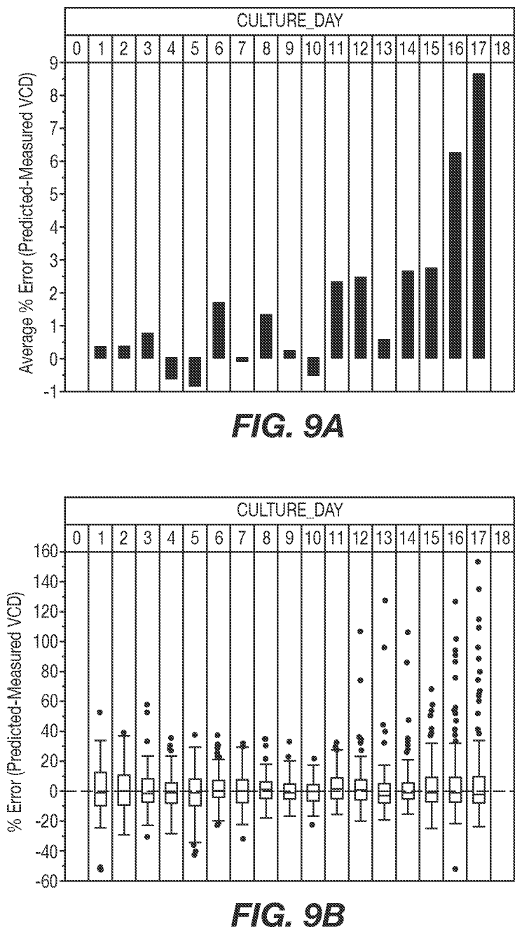

The percent error in VCD prediction is centered around zero, and varies by culture day, on average between 0 and 9%. FIGS. 9A-B illustrate that percent error in VCD prediction is below 10% on average.

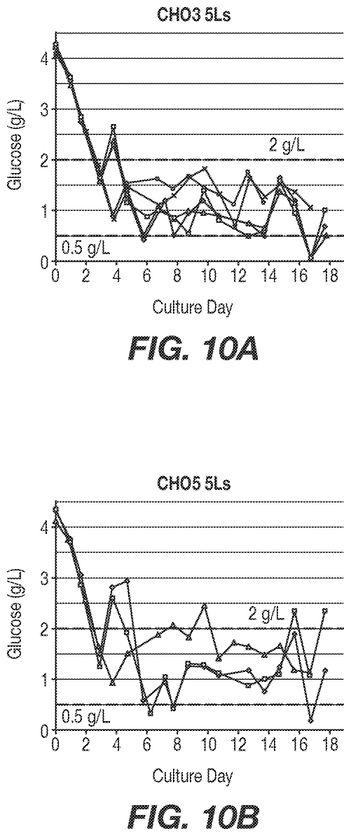

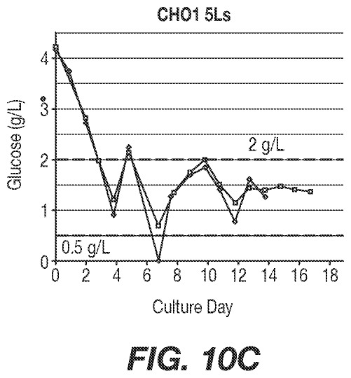

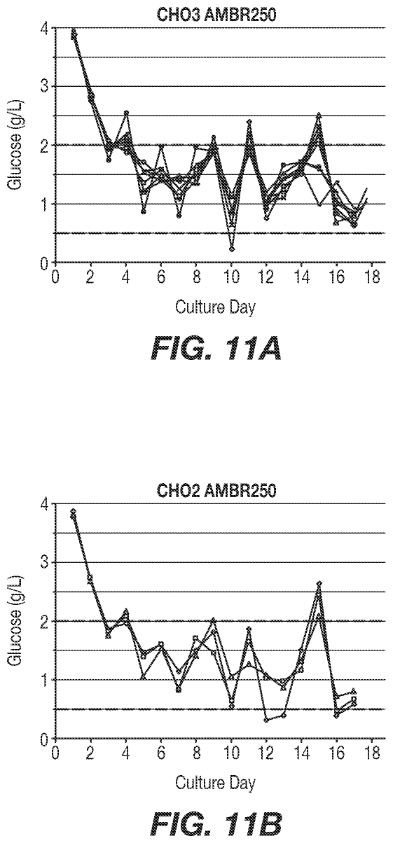

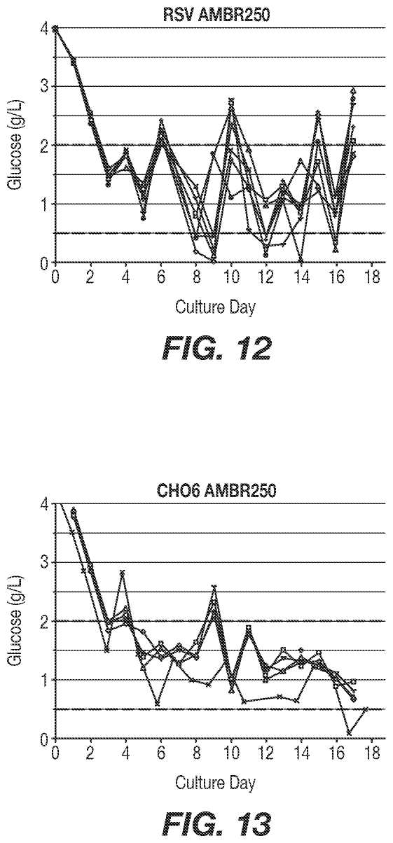

Glucose algorithm performance may be evaluated in 5 L, as illustrated in FIGS. 10A-C, and AMBR250, as illustrated in FIGS. 11A-B, FIG. 12 and FIG. 13, bioreactors with five cell lines (CHO1, CHO2, CHO3, CHO4, CHO5, and CHO6), as well as different high titer feeds (JMF-1, JMF-5, and JaMS). Residual glucose may be measured approximately one day after feeding, and the data demonstrate that the algorithm controlled glucose levels between 0.5 and 2 g/L in most cases.

In another embodiment, Equation 2 to Equation 4 may be substituted with the following equations. For example, the Predicted Glucose Consumption_{d+1} may be defined according to Equation 2-1. The algorithm assumes a time of 1 day.

Predicted Glucose Consumption_{d+1}=Glucose Consumption Rate_{d}*Predicted Fold Change in VCD_{d+1}*1 day [2-1]

The Glucose Consumption Rate_{d} may be defined according to Equation 3-1.

$$Glucose Consumption {Rate}_{d} = \frac{Glucose {Target}_{d - 1} - Measured {Glucose}_{d}}{Time Elapsed Between {Samples}_{d - ( d - 1 )}} \qquad [ 3 - 1 ]$$

The Predicted Fold Change in VCD_{d+1} may be defined in Equation 4-1. The algorithm assumes a time of 1 day.

$$Predicted Fold Change in {VCD}_{d + 1} = \frac{Median Expected \frac{Δ {IVCD}_{d + 1}}{Δ {IVCD}_{d}}}{Median Expected \frac{Δ {IVCD}_{d + 1}}{{VCD}_{d + 1}}} * 1 day \qquad [ 4 - 1 ]$$

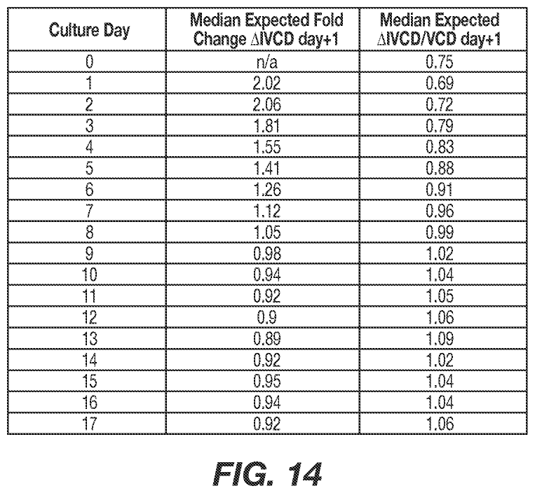

The values in Equation 4-1 may be calculated from a database of Janssen cell lines with High Titer media and feeds, as shown in FIG. 14. The database may be comprised of 162 5 L bioreactor runs. Six cell lines with varied phenotypes may be included, namely CHO1, CHO2, CHO3, CHO4, CHO5, and CHO6.

The Predicted VCD_{d+1} of Equation 5 may be defined as the Predicted Change in VCD_{d+1} of Equation 4-1 multiplied by ΔIVCD_{d}.

Residual glucose may be measured approximately one day after feeding, and the data demonstrate that the algorithm controlled glucose levels between 0.5 and 2 g/L in most cases.

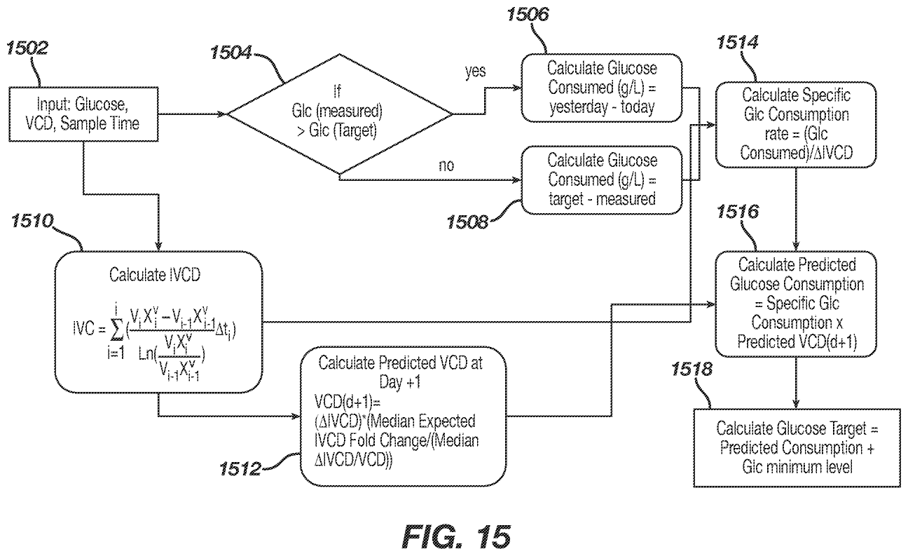

Glucose may be typically controlled between 0.5 g/L and 2 g/L. FIG. 15 illustrates an example flow process of calculating glucose target. At 1502, residual glucose measurement, VCD and sample time may be received as inputs. At 1504, the processor may determine whether the residual amount of glucose is greater than the target glucose. If yes, at 1506, a consumed amount of glucose is determined by determining an amount of glucose that is consumed between a previous day and a present day during the cell culture process. If no, at 1508, the consumed amount of glucose is determined based on a difference between a predetermined glucose target and the residual amount of glucose. At 1510, an integrated viable cell density IVCD is calculated. At 1512, predetermined viable cell density for a following day is calculated based on the integrated viable cell density. At 1514, a specific glucose consumption rate is calculated by dividing the consumed amount of glucose by ΔIVCD. At 1516, a predicted glucose consumption amount is calculated by multiplying the specific glucose consumption rate by predetermined viable cell density for the following day. At 1518, the glucose target is calculated by summing the predicted glucose consumption amount and a predetermined glucose minimum amount.

### 2.0 Variable Glucose Feed Sheet Instructions

### 2.1 Instructions for AMBR 250 Bioreactors

### 2.1.1 First Step

The feed sheet can be found on the Resources for High Titer Implementation SharePoint site. This site is linked on the API-LM SharePoint Page.

### 2.1.2 Second Step

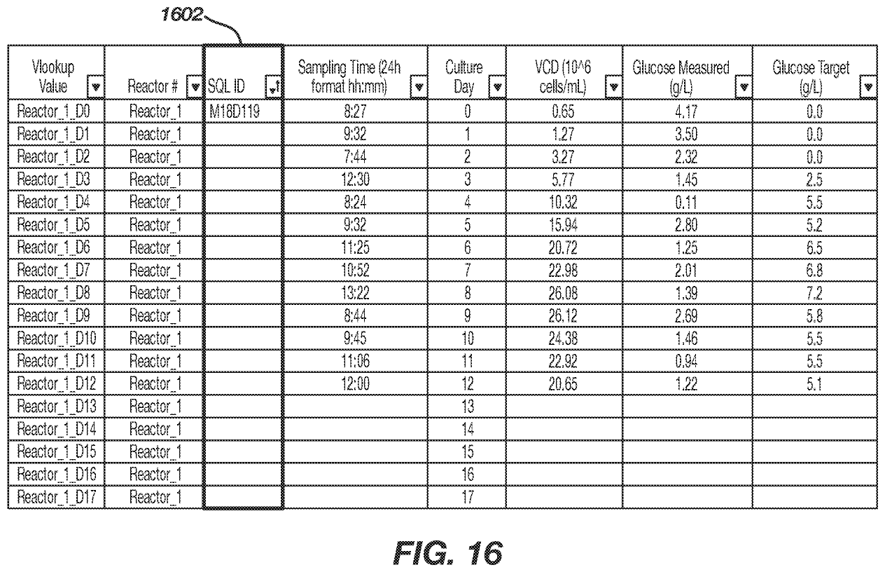

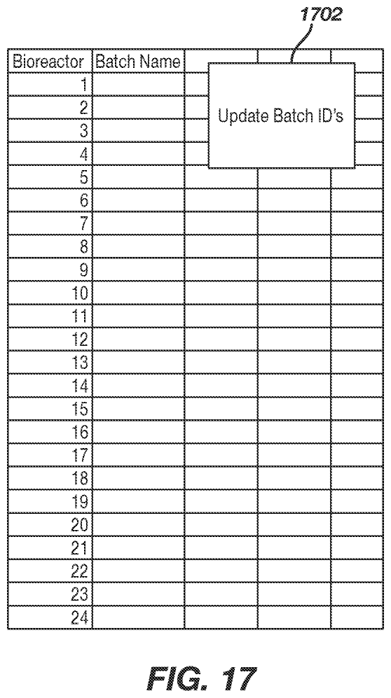

Before the start of run, enter SQL IDs 1602 for each bioreactor and culture day, as illustrated in FIG. 16. To add Bioreactor SQL ID's using the files macros follow instructions as illustrated in FIG. 17. First, on the “Batches” tab, enter bioreactor ID's into column B under Batch Name. Second, press the “Update Batch ID's” button 1702 when finished. Third, bioreactor ID's are populated in the “Inputs and Feed Targets” sheet.

### 2.1.3 Third Step

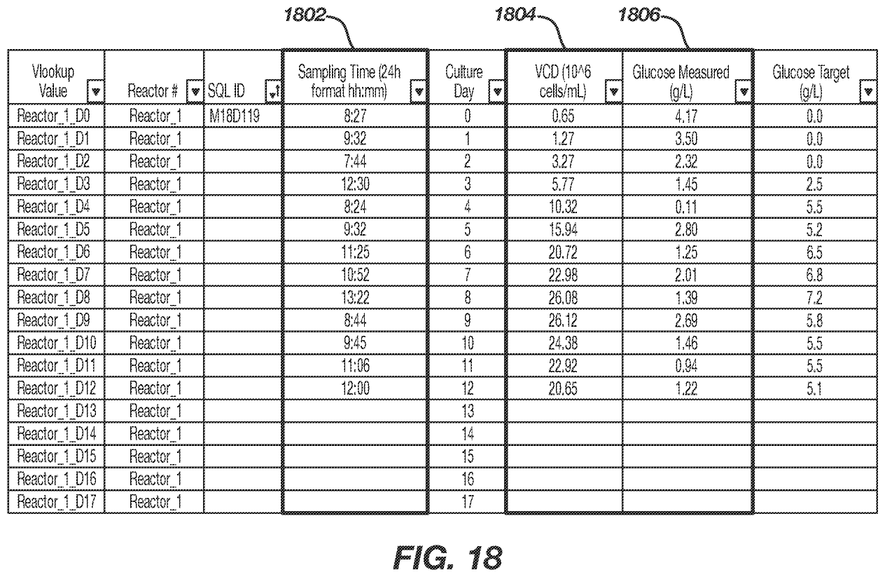

For each AMBR250 bioreactor and culture day, enter the sampling time 1802 (in 24 hour format hh:mm), measured VCD 1804 (10\hat{ }6 cells/mL) and glucose concentration 1806 in the bioreactor before feeding (g/L) either manually or using macros, as illustrated in FIG. 18.

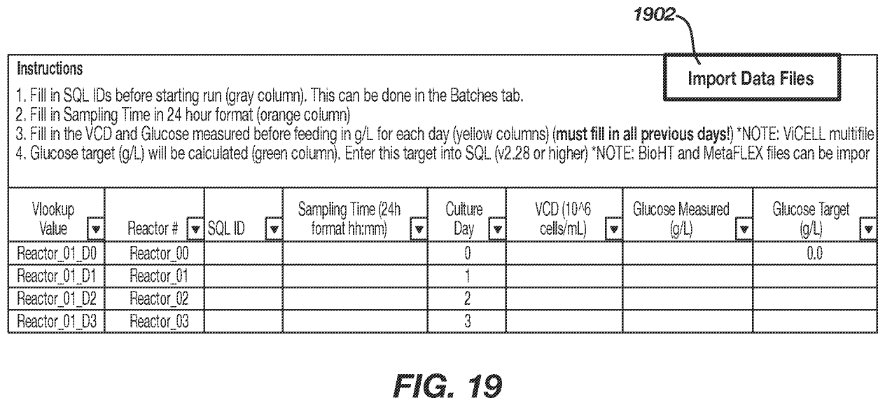

Macros within the excel file allow file imports for external readings from Vi-CELL, BioHT, MetaFLEX, and AMBR250 instruments. The sample naming requirements detailed below need to be followed to accurately import values for each instrument file type. Vi-CELL multi-files or AMBR250 data tables can be imported for VCD measurements. MetaFLEX or BioHT files can be imported for glucose measurements, as illustrated in FIG. 19.

### 2.1.3.1 Vi-CELL Multi-File

First, bioprocesses should be created for each bioreactor prior to taking sampling readings. Second, the “Comments” section should be used to indicate the culture day with the nomenclature D #(Culture day 1 example=D1). Third, all readings are to be saved to a single excel multi-file. Multi-files are created through the Vi-CELL software.

### 2.1.3.2 BioHT Text Files

BioHT sample name nomenclature is to follow the Malvern API-LM format; “Bioreactor ID”_D #.

### 2.1.3.3 MetaFLEX Files

First, the “Bioreactor ID” is entered on the “Patient ID” entry on the MetaFLEX. Second, the culture day number is entered on the “Patient Name” entry on the MetaFLEX (Only enter the #, not D #).

### 2.1.3.4 AMBR250 Vi-CELL Values

Vi-CELL values from the AMBR250 are exported into daily tables through the AMBR250 software. The table contains only the bioreactor, “batch name”, and “viable cell density” columns from the AMBR250 software.

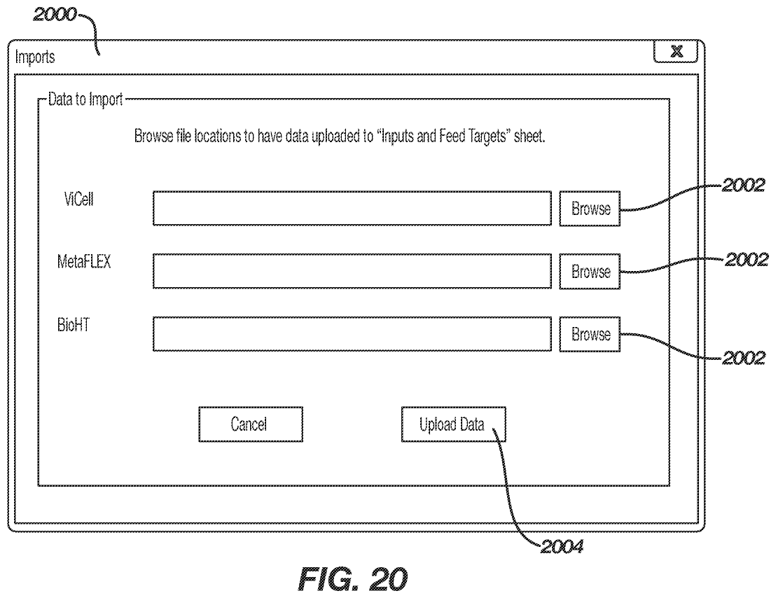

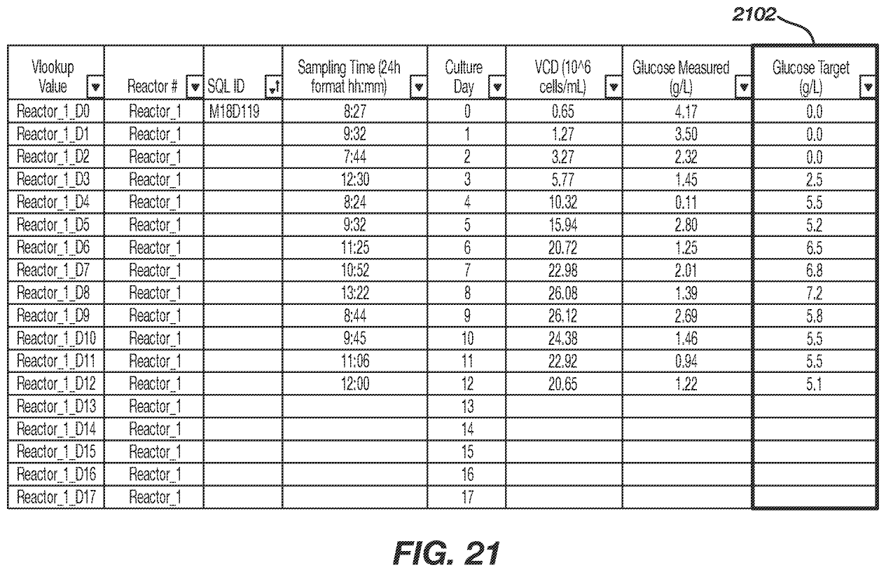

After pressing the “Import Data Files” button 1902 as illustrated in FIG. 19 for the “Inputs and Feed Targets” sheet, a “Data to Import” window 2000 appears, as shown in FIG. 20. Files to add cell counts and glucose values can be selected by pressing the appropriate “Browse” button 2002 and navigating to the desired file, such as ViCELL multi-files or AMBR250 ViCELL CSV file for the ViCell, MetaFLEX CSV files for MetaFlex, and BioHT text files for BioHT. Press the “Upload Data” button 2004 after files have been selected. VCD and glucose values are populated in the “Inputs and Feed Targets” sheet.

For AMBR250, the pH and sampling times are populated when using the MetaFLEX import.

Culture days are not exported from the AMB250. When importing AMBR250 Vi-CELL files, an additional dialog window will open asking for a culture day. Enter the numeric culture day in this window (culture day 1 example=1).

### 2.1.4 Fourth Step

The feed sheet will calculate a glucose target (g/L) for each AMBR250 vessel, which the operator can enter into SQL under the ‘Glucose_Target’ column 2102 and can be either manually entered into the AMBR250 tables or semi-automatically entered into the AMBR250 tables using macros, as illustrated in FIG. 21.

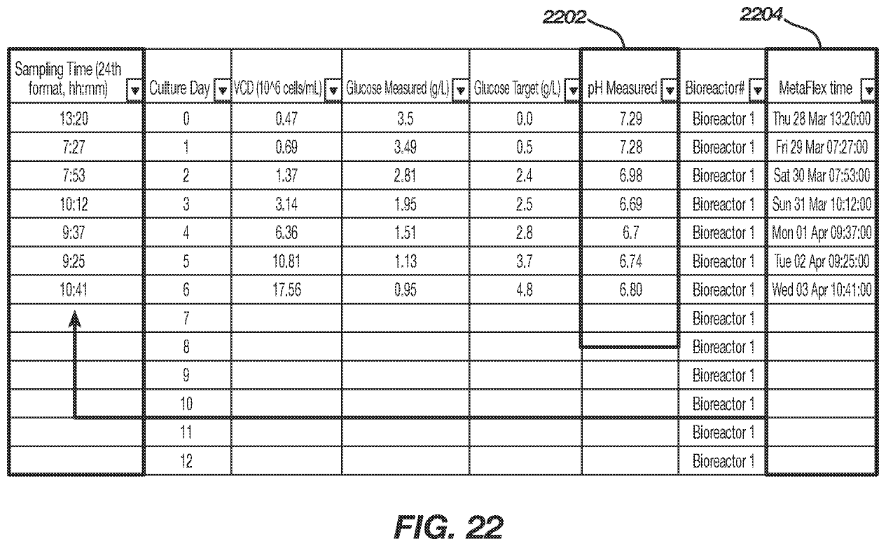

For AMBR250, when importing a MetaFLEX file the pH 2202 and time 2204 are populated into the glucose feed sheet, as illustrated in FIG. 22. This can be later used to import the daily pH offsets to the AMBR250. This only works for the MetaFLEX import, not the BioHT import. The pH is included in the illustration of FIG. 22 to make operation of some of the micro-scale bioreactors easier. The online pH measured by the controller may be verified with an offline pH measurement. If these are different, the online measurement is corrected. By including pH in the sheet, user can easily import off-line measurement values to do “pH offset”. The pH is not needed for glucose target calculation.

### 2.1.5 Fifth Step

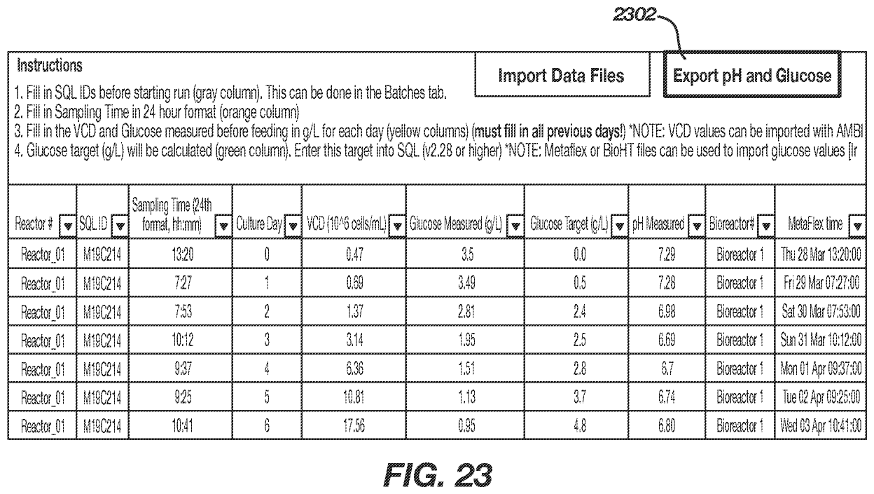

By using the “Export pH and Glucose” macro 2302, as illustrated in FIG. 23, the glucose sheet will export two CSV files: a pH offline measurements file and a glucose measured/glucose target file for a chosen day. Save the two tables to a flash drive.

If MetaFLEX is not being used to measure the glucose/pH then the pH file is blank.

### 2.1.6 Sixth Step

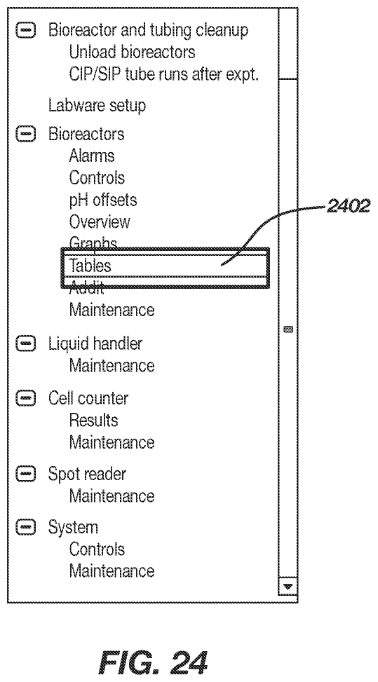

To enter glucose target values into the AMBR software, click on the ‘Tables’ tab 2402 on the left-hand navigation bar, as illustrated in FIG. 24.

### 2.1.7 Seventh Step

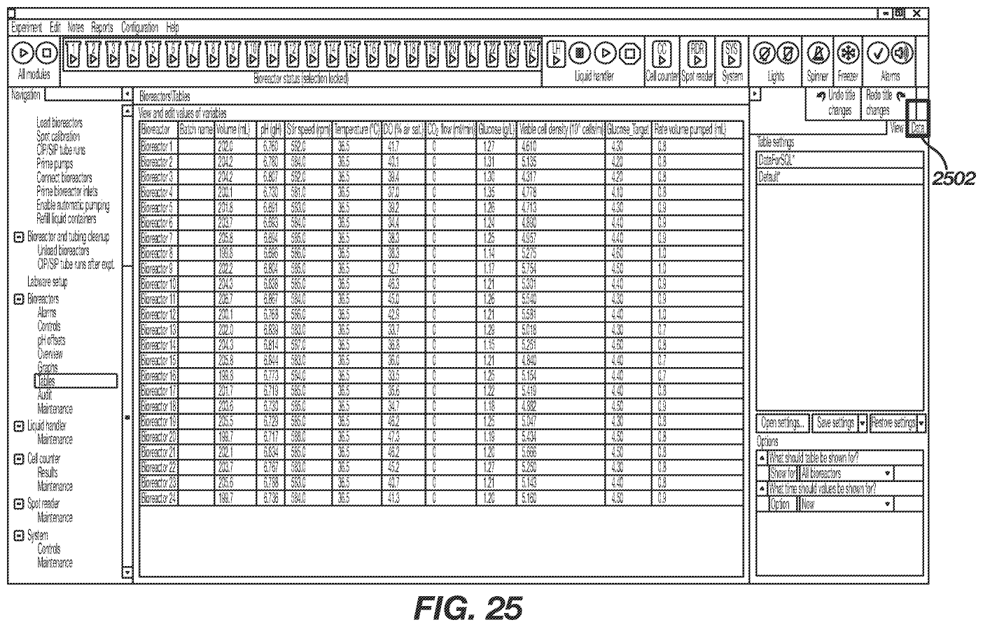

Then, on the right-hand side of the screen, click on the ‘Data’ tab 2502, as illustrated in FIG. 25.

### 2.1.8 Eighth Step

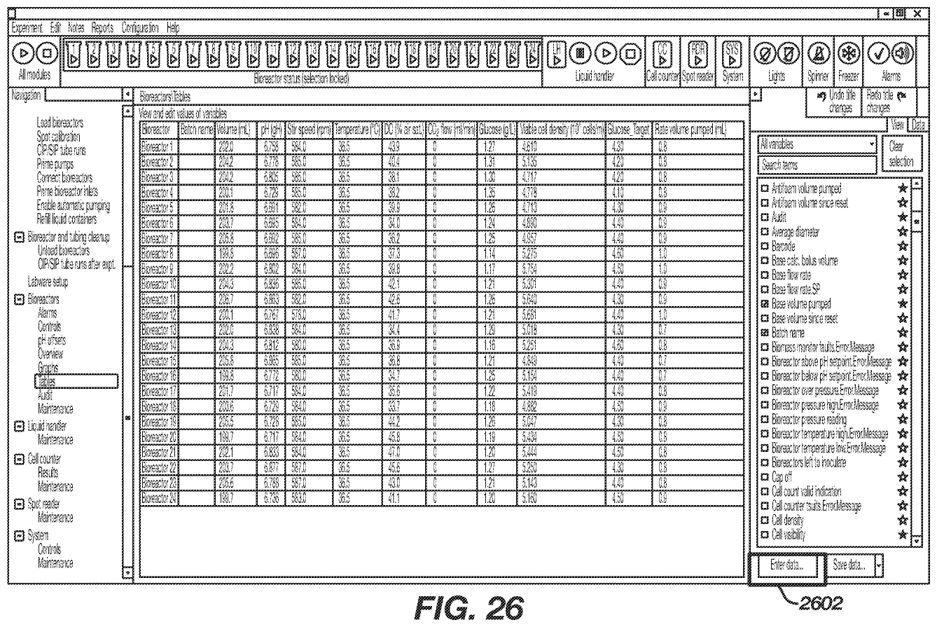

Next, click on ‘Enter data . . . ’ button 2602 on the bottom right of the screen, as illustrated in FIG. 26. A dialogue box will pop up. (2 methods—using the export macro or manually entering the data).

### 2.1.9 Ninth Step

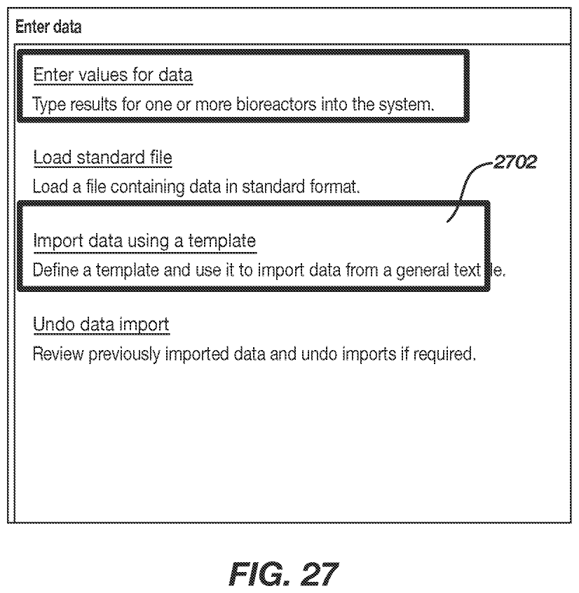

In the dialogue box, if using the Semi-automated Macros Method: ‘click’ “Import Data using a template” link 2702 (usable if the Glucose file may be exported from step 2.1.5), as illustrated in FIG. 27. If using the Manual method: ‘click’ “Enter values for data”.

### 2.1.10 Tenth Step

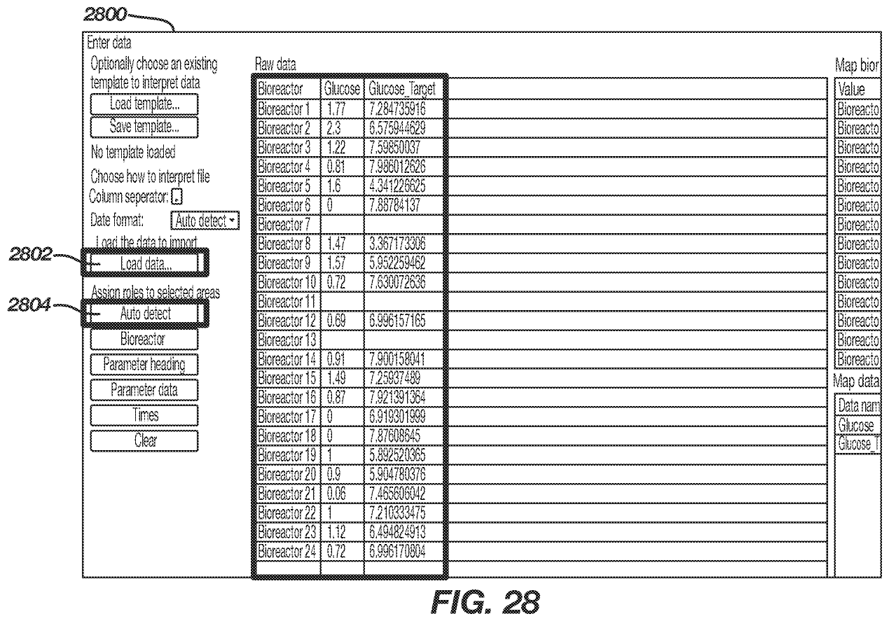

If using the manual approach, skip to step 2.1.12. If using the semi-automated macros method, after selecting “Import Data using a template” link 2702 a new dialogue box 2800 will appear, as illustrated in FIG. 28. On the left-hand side select “Load Data” button 2802. Once the Table is loaded ‘highlight’ all the cells (Click the first cell and drag to the last cell), then select the “Auto Detect” button 2804.

### 2.1.11 Eleventh Step

The cells are populated with a color code, dependent to the type of data found in each cell (Bioreactor, Data heading, and Data parameters). If all off the cells are correctly code ‘Click’ “Save Data”.

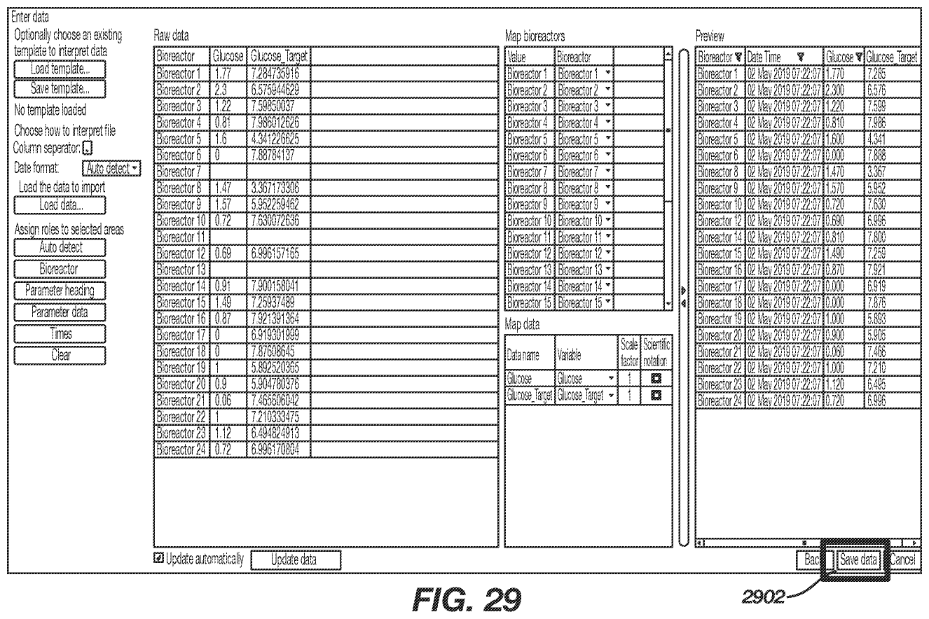

If using the MetaFLEX instrument, the variable glucose algorithm feed sheet can export the measured pH in the second saved file. On the left-hand navigation bar select the “pH offset” and select all the reactors, then select the “Calibrate pH offsets”. In the dialogue box, select the “Import Data File” on the lower left of the screen a select the ‘exported pH file’. When the data is populated, remove any inactivated bioreactors and select “Save Data” button 2902 as illustrated in FIG. 29.

### 2.1.12 Twelfth Step

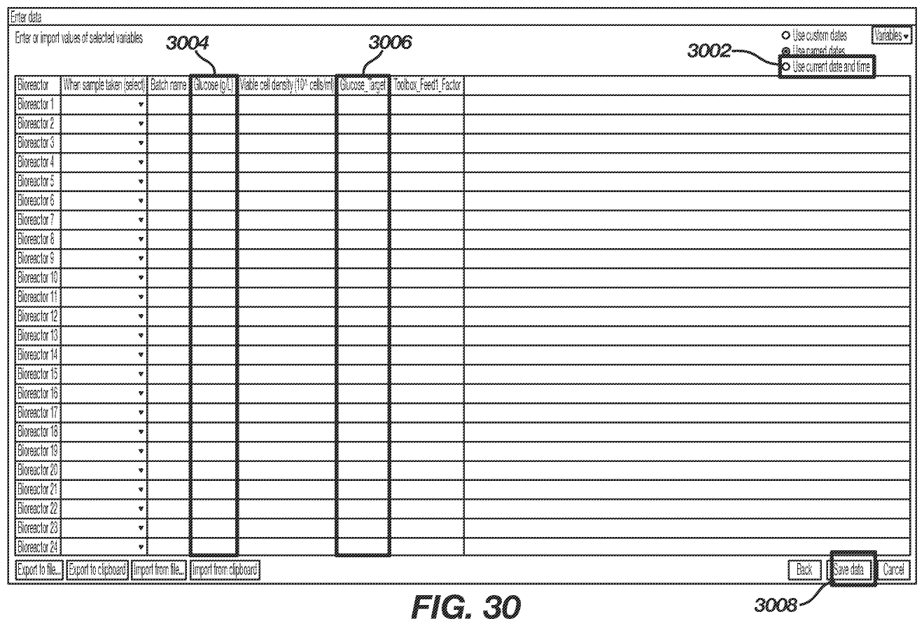

If the export button from 2.1.5 did NOT work “Enter Value for Data”: In upper right corner of the next window, click ‘use current date and time’ 3002 as illustrated in FIG. 30. For each AMBR bioreactor, enter measured glucose values (g/L) 3004 and glucose target values (g/L) 3006 into the table.

### 2.1.13 Thirteenth Step

Finally, click ‘Save data’ 3008 in the bottom right hand corner of the screen as illustrated in FIG. 30.

### 2.2 Instructions for 5 L Bioreactors

### 2.2.1 First Step

The feed sheet can be found on the Resources for High Titer Implementation SharePoint site. This site is linked on the API-LM SharePoint Page.

### 2.2.2 Second Step

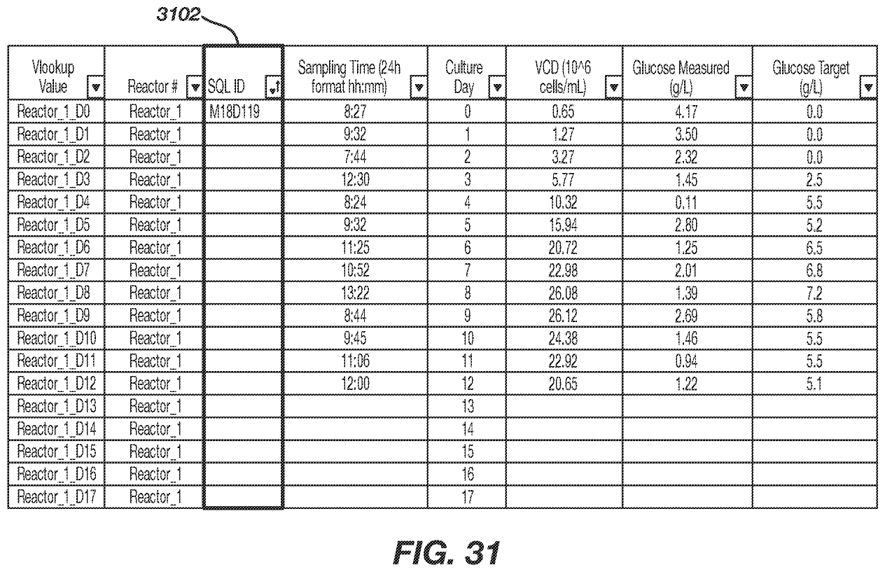

Before the start of run, enter SQL Bioreactor ID's for each reactor and culture day. SQL Bioreactor s 3102 can be added using imbedded macros, as illustrated in FIG. 31. See section 2.1.2 for macro use to populate bioreactor ID's.

### 2.2.3 Third Step

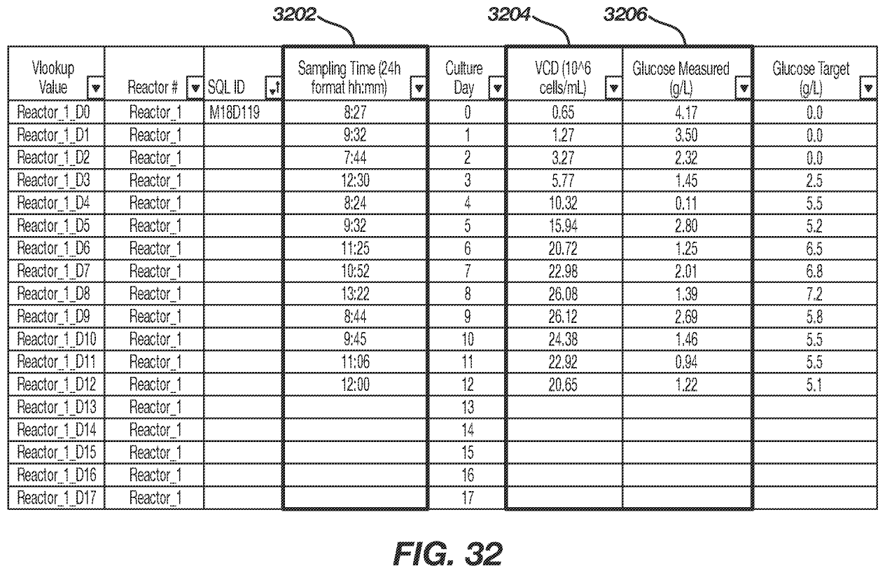

For each 5 L bioreactor and culture day, enter the sampling time 3202 (in 24 hour format hh:mm), measured VCD 3204 (10\hat{ }6 cells/mL) and glucose concentration 3206 in the bioreactor before feeding (g/L), as illustrated in FIG. 32.

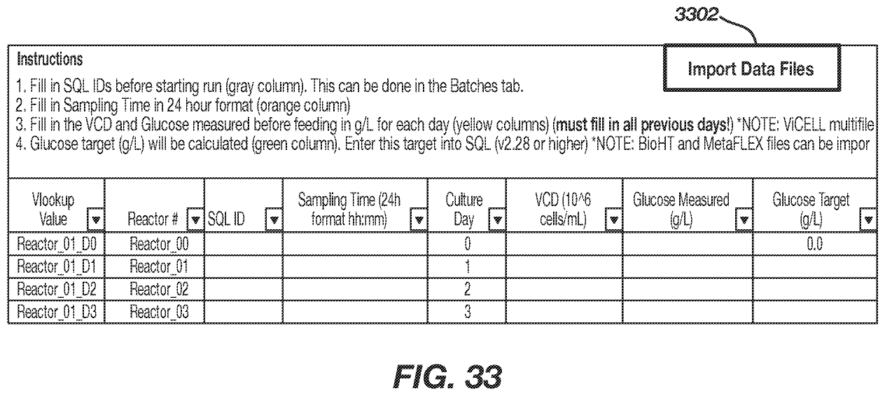

Macros within the excel file allow file imports 3302 for external readings from Vi-CELL, BioHT, MetaFLEX, and AMBR250 instruments, as illustrated in FIG. 33. The sample naming requirements detailed below need to be followed to accurately import values for each instrument file type. Vi-CELL multi-files or AMBR250 data tables can be imported for VCD measurements. MetaFLEX or BioHT files can be imported for glucose measurements.

### 2.2.3.1 Vi-CELL Multi-File

Bioprocesses should be created for each bioreactor prior to taking sampling readings. The “Comments” section should be used to indicate the culture day with the nomenclature D #(Culture day 1 example=D1). All readings are to be saved to a single excel multi-file. Multi-files are created through the Vi-CELL software.

### 2.2.3.2 BioHT Text Files

BioHT sample name nomenclature is to follow the Malvern API-LM format; “Bioreactor ID”_D #.

### 2.2.3.3 MetaFLEX Files

The “Bioreactor ID” is entered on the “Patient ID” entry on the MetaFLEX. The culture day number is entered on the “Patient Name” entry on the MetaFLEX (Only enter the #, not D #)

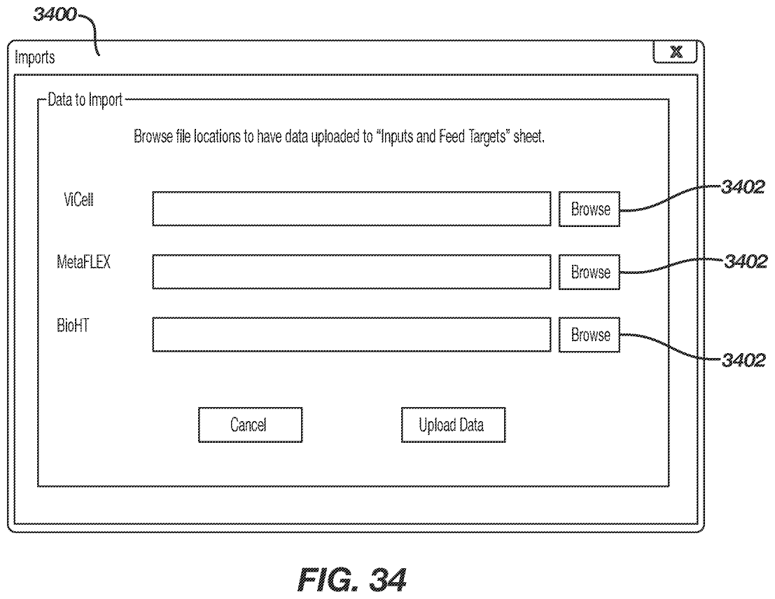

After pressing the “Import Data Files” button for the “Inputs and Feed Targets” sheet, a Data to Import window 3400 appears, as illustrated in FIG. 34. Files to add cell counts and glucose values can be selected by pressing the appropriate “Browse” button 3402 and navigating to the desired file, such as ViCELL multi-files ViCELL CSV for ViCell, MetaFLEX CSV files for MetaFLEX, and BioHT text files for BioHT. Press the “Upload Data” button after files have been selected. VCD and glucose values are populated in the “Inputs and Feed Targets” sheet.

### 2.2.4 Fourth Step

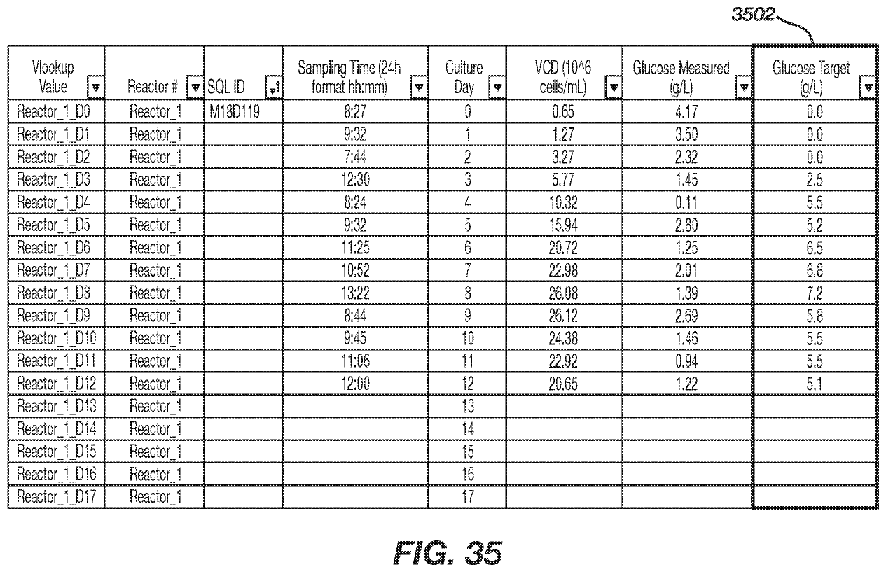

The feed sheet will calculate a glucose target (g/L) for each 5 L vessel, which the operator will enter into SQL under the ‘Glucose Target’ column 3502, as illustrated in FIG. 35.

### 2.2.5 Fifth Step

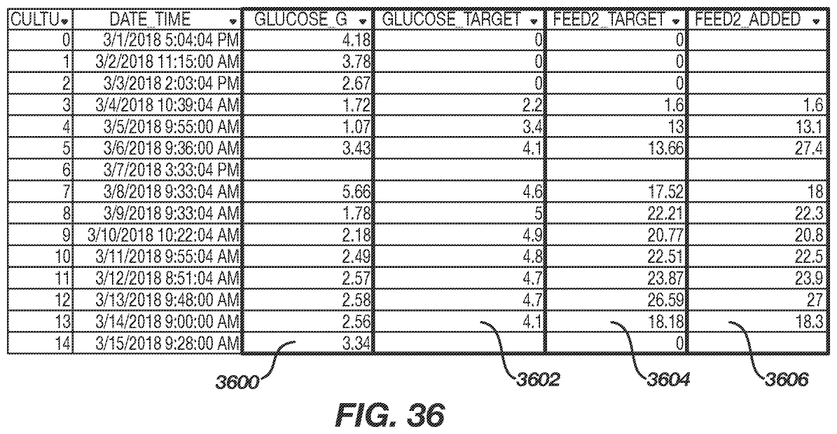

In SQL (v 2.28 or greater), enter the measured glucose (g/L) into the ‘Glucose_G’ column 3600 and the glucose target into the ‘Glucose_Target’ column 3602. The volume of glucose to add to the bioreactor is calculated in the ‘Feed2_Target’ column 3604. The volume of glucose 3606 actually fed to the bioreactor is recorded, as illustrated in FIG. 36.

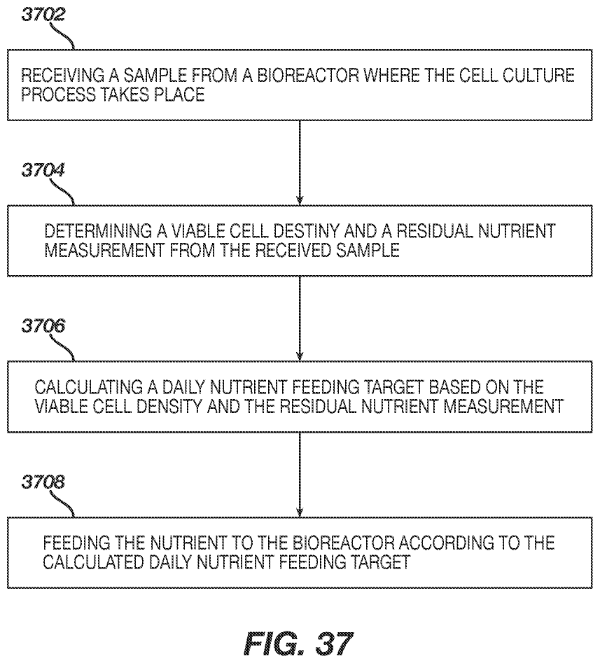

FIG. 37 is an example flow chart illustrating a process of controlling a nutrient feed in a cell culture process. At 3702, a sample may be received from a bioreactor comprising a cell culture. At 3704, a viable cell density and a residual nutrient measurement may be determined from the received sample. At 3706, a daily nutrient feeding target may be calculated based on the viable cell density and the residual nutrient measurement. At 3708, the nutrient may be fed to the bioreactor according to the calculated daily nutrient feeding target.

In one embodiment, the process may also include maintaining a daily residual nutrient concentration in the bioreactor within a predetermined range.

In one embodiment, the daily nutrient feeding target may be recalculated based on the viable cell density and the residual nutrient measurement on a daily basis.

In one embodiment, the nutrient may be selected from glucose, glutamate, galactose, lactate, and glutamine.

In one embodiment, the nutrient may include one or more monosaccharides.

In one embodiment, the residual nutrient measurement may include assaying a nutrient concentration in the bioreactor.

In one embodiment, the residual nutrient measurement may include performing one or more of offline nutrient measurement and inline nutrient measurement.

In one embodiment, the residual nutrient measurement may be performed by one or more of the following: a NovaFlex device and a Raman Probe.

In one embodiment, the bioreactor may be one or more of the following: a Chinese hamster ovary (CHO) cell bioreactor, and a 5 L bioreactor. Other mammalian cell types that may be used in manufacturing biologics besides CHO, including recombinant cells and the like. Non-limiting examples of such mammalian cell types include HEK, 293 and PerC6. This process may be also used for other non-mammalian cell types, such as, for example, yeast and bacteria.

In one embodiment, cells in the bioreactor may be mammalian cells.

In one embodiment, the cells are CHO cells.

In one embodiment, the daily nutrient feeding target may be calculated based at least in part on a global average consumption value and a growth profile predetermined in advance from multiple runs of the bioreactor from at least 6 cell lines.

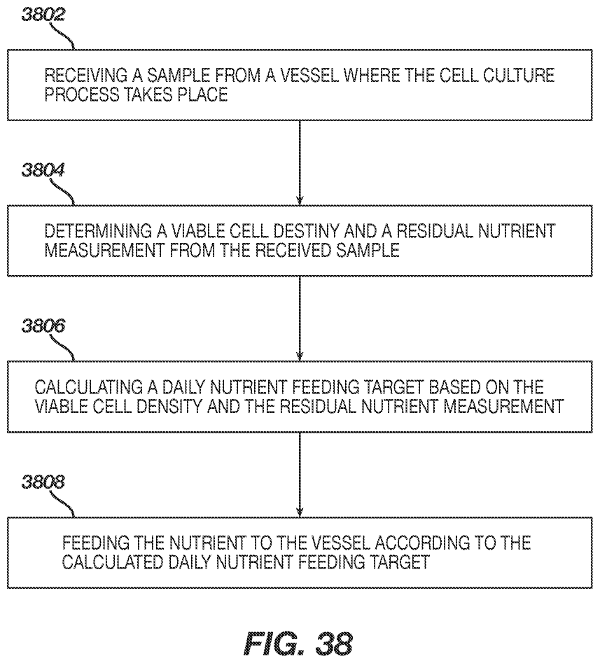

FIG. 38 is another example flow chart illustrating a process of controlling a nutrient feed in a cell culture process. At 3802, a sample may be received from a vessel comprising a cell culture. At 3804, a viable cell density and a residual nutrient measurement may be determined from the received sample. At 3806, a daily nutrient feeding target may be calculated based on the viable cell density and the residual nutrient measurement. At 3808, the nutrient may be fed to the vessel according to the calculated daily nutrient feeding target. In one embodiment, the vessel may be a flask.

In one embodiment, the nutrient may be selected from glucose, glutamate, galactose, lactate, and glutamine.

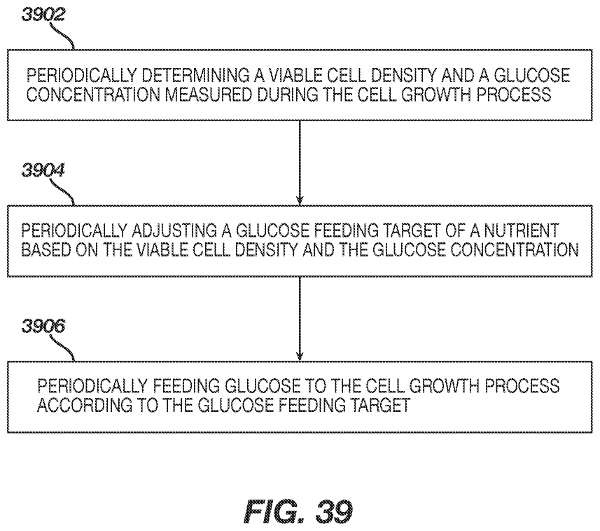

FIG. 39 is an example flow chart illustrating a process of balancing a glucose feed in a cell growth process. At 3902, a viable cell density and a glucose concentration measured during the cell growth process may be periodically determined. At 3904, a glucose feeding target of a nutrient may be periodically adjusted based on the viable cell density and the glucose concentration. At 3906, glucose may be periodically fed to the cell growth process according to the glucose feeding target.

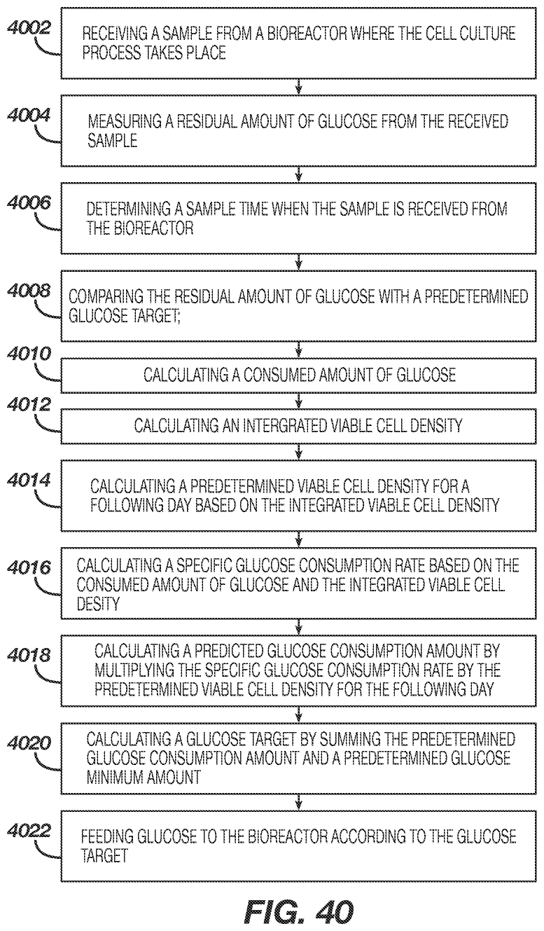

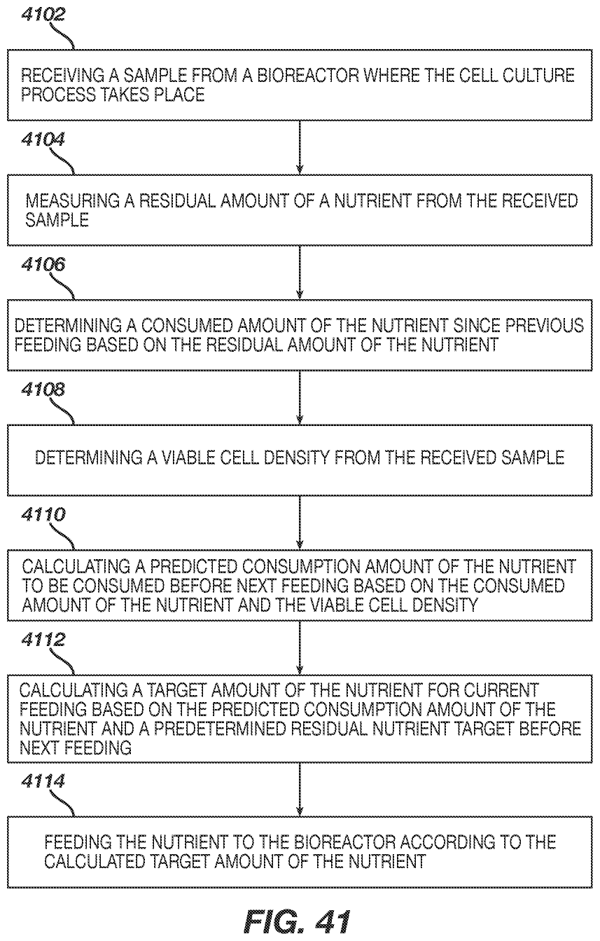

FIG. 40 is an example flow chart illustrating a process of controlling a glucose feed in a cell culture process. At 4002, a sample may be received from the production rector comprising a cell culture. At 4004, a residual amount of glucose may be measured from the received sample. At 4006, a sample time when the sample is received from the production reactor may be determined. At 4008, the residual amount of glucose may be compared with a predetermined glucose target. At 4010, a consumed amount of glucose may be calculated. For example, the consumed amount of glucose may be determined by determining an amount of glucose that is consumed between a previous day and a present day during the cell culture process when the residual amount of glucose is greater than the predetermined glucose target. In another example, the consumed amount of glucose may be determined based on a difference between the predetermined glucose target and the residual amount of glucose when the residual amount of glucose is not greater than the predetermined glucose target.

At 4012, an integrated viable cell density may be calculated. At 4014, a predetermined viable cell density for a following day may be calculated based on the integrated viable cell density. At 4016, a specific glucose consumption rate may be calculated based on the consumed amount of glucose and the integrated viable cell density. At 4018, a predicted glucose consumption amount may be calculated by multiplying the specific glucose consumption rate by the predetermined viable cell density for the following day. At 4020, a glucose target may be calculated by summing the predetermined glucose consumption amount and a predetermined glucose minimum amount. At 4022, glucose may be fed to the production reactor according to the glucose target.

In one embodiment, the feeding may take place on a daily basis.

FIG. 41 is an example flow chart illustrating a process of modulating an amount of glycation of an agent in a cell culture process. At 4102, a sample may be received from a production reactor comprising a cell culture. At 4104, a residual amount of a nutrient may be measured from the received sample. At 4106, a consumed amount of the nutrient since previous feeding may be determined based on the residual amount of the nutrient. At 4108, a viable cell density may be determined from the received sample. At 4110, a predicted consumption amount of the nutrient to be consumed before next feeding may be calculated based on the consumed amount of the nutrient and the viable cell density. At 4112, a target amount of the nutrient for current feeding may be calculated based on the predicted consumption amount of the nutrient and a predetermined residual nutrient target before next feeding. At 4114, the nutrient may be fed to the bioreactor according to the calculated target amount of the nutrient.

In one embodiment, a predicted viable cell density between the current feeding and the next feeding may be determined based at least in part on the determined viable cell density. A nutrient consumption rate may be determined based at least in part on the consumed amount of the nutrient. The predicted consumption rate may be calculated based on the predicted viable cell density and the nutrient consumption rate.

In one embodiment, the feeding may take place on a daily basis.

In one embodiment, the nutrient may be selected from glucose, glutamate, galactose, lactate, and glutamine.

In one embodiment, the nutrient may include one or more monosaccharides.

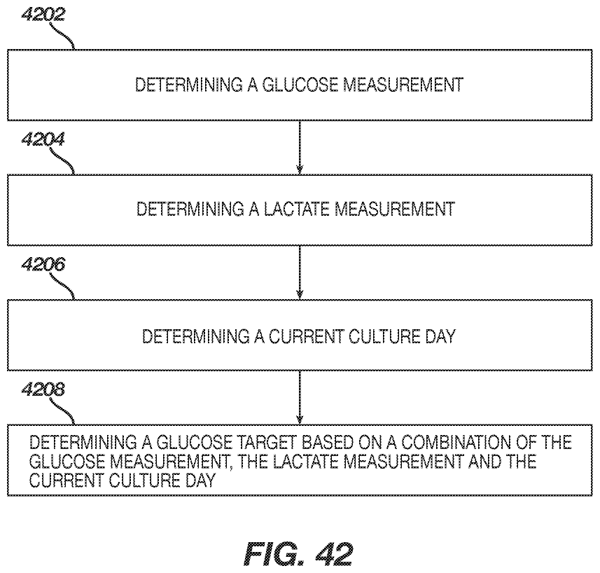

FIG. 42 is an example flow chart illustrating a process of controlling a glucose feed in a cell culture process. At 4202, a glucose measurement may be determined. At 4204, a lactate measurement may be determined. At 4206, a current culture day may be determined. At 4208, a glucose target may be determined based on a combination of the glucose measurement, the lactate measurement and the current culture day.

In one embodiment, the glucose target may be 5 g/L, when the glucose measurement is less than 1 g/L, the lactate measurement is less than 1 g/L, and the current culture day is day 5.

In one embodiment, the glucose target may be 4.5 g/L when the glucose measurement is less than 1 g/L, the lactate measurement is greater than 1 g/L and less than 3 g/L, and the current culture day is day 5.

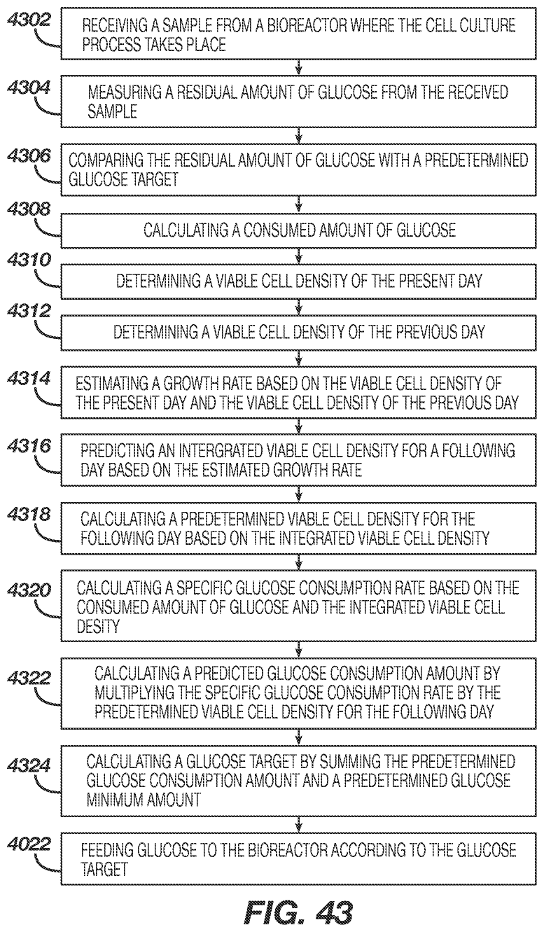

FIG. 43 is another example flow chart illustrating a process of controlling a glucose feed in a cell culture process. At 4302, a sample may be received from the production reactor comprising a cell culture. At 4304, a residual amount of glucose may be measured from the received sample. At 4306, the residual amount of glucose may be compared with a predetermined glucose target. At 4308, a consumed amount of glucose may be calculated. For example, the consumed amount of glucose may be determined by determining an amount of glucose that is consumed between a previous day and a present day during the cell culture process when the residual amount of glucose is greater than the predetermined glucose target. In another example, the consumed amount of glucose may be determined based on a difference between the predetermined glucose target and the residual amount of glucose when the residual amount of glucose is not greater than the predetermined glucose target. At 4310, a viable cell density of the present day may be determined. At 4312, a viable cell density of the previous day may be determined. At 4314, a growth rate may be estimated based on the viable cell density of the present day and the viable cell density of the previous day. At 4316, an integrated viable cell density for a following day may be predicted based on the estimated growth rate. At 4318, a predetermined viable cell density for the following day may be calculated based on the integrated viable cell density. At 4320, a specific glucose consumption rate may be calculated based on the consumed amount of glucose and the integrated viable cell density. At 4322, a predicted glucose consumption amount may be calculated by multiplying the specific glucose consumption rate by the predetermined viable cell density for the following day. At 4324, a glucose target may be calculated by summing the predetermined glucose consumption amount and a predetermined glucose minimum amount. At 4326, glucose may be fed to the production reactor according to the glucose target.

Below is a list of abbreviations and definitions.

d may refer to current cell culture day.

d+1 may refer to next cell culture day.

d−1 may refer to previous cell culture day.

VCD may refer to viable cell density (cells/mL).

IVCD may refer to integrated viable cell density (cells/mL*day).

ΔIVCD_{d} may refer to a change in IVCD from day d−1 to d (cells*day/mL).

VCD_{d+1} may refer to VCD on day d+1.

ΔIVCD_{d+1} may refer to a change in IVCD from day d to d+1 (cells*day/mL).

IQR may refer to interquartile range.

Glucose Target_{d} may refer to target concentration of glucose to feed reactor (g/L).

Glucose Consumption Rate_{d} may refer to glucose consumed per day from time d−1 to time d ((g/L)/day).

Specific Glucose Consumption Rate_{d} may refer to glucose consumed per cell-day from time d−1 to time d (pg/(cell*day)).

Residual Glucose Target_{d+1} may refer to desired theoretical concentration of residual glucose in the reactor at time d+1 (g/L).

Predicted Glucose Consumption_{d+1} may refer to predicted glucose consumed at time d+1 (g/L).

Predicted Fold Change in VCD_{d+1} may refer to predicted fold change in VCD from time d to time d+1, derived from database. Assumption is made that 1 day has elapsed (unitless).

Predicted Glucose Consumption_{d+1} may refer to concentration of glucose predicted to be consumed by the cell culture from time d to time d+1 (g/L).

Measured Glucose_{d} may refer to the concentration of glucose measured in the bioreactor for the current day d.

ΔIVCD_{d+1}/VCD_{d+1} may refer to fold difference between ΔIVCD and VCD from d to d+1. Equal to 1 if ΔIVCD=VCD.

ΔIVCD_{d+1}/ΔIVCD_{d} may refer to fold change in ΔIVCD from d to d+1.

Measured VCD_{d+1} may refer to VCD measured on d+1.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

Certain implementations of the disclosed technology are described above with reference to block and flow diagrams of systems and methods and/or computer program products according to example implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, or may not necessarily need to be performed at all, according to some implementations of the disclosed technology.

These computer program instructions may also be stored in a computer-readable memory that can direct a computer or other programmable data processing apparatus to function in a particular manner, such that the instructions stored in the computer-readable memory produce an article of manufacture including instruction means that implement one or more functions specified in the flow diagram block or blocks.

Implementations of the disclosed technology may provide for a computer program product, comprising a computer-usable medium having a computer-readable program code or program instructions embodied therein, said computer-readable program code adapted to be executed to implement one or more functions specified in the flow diagram block or blocks. The computer program instructions may also be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions and program instruction means for performing the specified functions. It will also be understood that each block of the block diagrams and flow diagrams, and combinations of blocks in the block diagrams and flow diagrams, can be implemented by special-purpose, hardware-based computer systems that perform the specified functions, elements or steps, or combinations of special-purpose hardware and computer instructions.

1. A method of controlling a nutrient feed in a cell culture process, comprising:
receiving a sample from a bioreactor comprising a cell culture;
determining a viable cell density and a residual nutrient measurement from the received sample;
calculating a daily nutrient feeding target based on the viable cell density and the residual nutrient measurement; and
feeding the nutrient to the bioreactor according to the calculated daily nutrient feeding target.

2. The method of claim 1, further comprising maintaining a daily residual nutrient concentration in the bioreactor within a predetermined range.

3. The method of claim 1 or claim 2, further comprising recalculating the daily nutrient feeding target based on the viable cell density and the residual nutrient measurement on a daily basis.

4. The method of any one of claims 1 to 3, wherein the nutrient is selected from glucose, glutamate, galactose, lactate, and glutamine.

5. The method of any one of claims 1 to 3, wherein the nutrient includes one or more monosaccharides.

6. The method of any one of claims 1 to 5, wherein the residual nutrient measurement comprises assaying a nutrient concentration in the bioreactor.

7. The method of any one of claims 1 to 5, wherein the residual nutrient measurement comprises performing one or more of offline nutrient measurement and inline nutrient measurement.

8. The method of any one of claims 1 to 5, wherein the residual nutrient measurement is performed by one or more of the following: a NovaFlex device and a Raman Probe.

9. The method of any one of claims 1 to 8, wherein the bioreactor is one or more of the following: a Chinese hamster ovary (CHO) cell bioreactor and a 5 L bioreactor.

10. The method of any one of claims 1 to 9, wherein cells in the bioreactor are mammalian cells.

11. The method of claim 10, wherein the cells are CHO cells.

12. The method of any one of claims 1 to 11, wherein the daily nutrient feeding target is calculated based at least in part on a global average consumption value and a growth profile predetermined in advance from multiple runs of the bioreactor from at least 6 cell lines.

13. A method of controlling a nutrient feed in a cell culture process, comprising:
receiving a sample from a vessel comprising a cell culture;
determining a viable cell density and a residual nutrient measurement from the received sample;
calculating a daily nutrient feeding target based on the viable cell density and the residual nutrient measurement; and
feeding the nutrient to the vessel according to the calculated daily nutrient feeding target.

14. The method of claim 13, wherein the vessel is a flask.

15. The method of claim 13 or claim 14, wherein the nutrient is selected from glucose, glutamate, galactose, lactate, and glutamine.

16. A method of balancing a glucose feed in a cell growth process, comprising:
periodically determining a viable cell density and a glucose concentration measured during the cell growth process;
periodically adjusting a glucose feeding target of a nutrient based on the viable cell density and the glucose concentration; and
periodically feeding glucose to the cell growth process according to the glucose feeding target.

17. A method of controlling a glucose feed in a cell culture process, comprising:
receiving a sample from a bioreactor comprising a cell culture;
measuring a residual amount of glucose from the received sample;
determining a sample time when the sample is received from the bioreactor;
comparing the residual amount of glucose with a predetermined glucose target;
calculating a consumed amount of glucose by:
determining the consumed amount of glucose by determining an amount of glucose that is consumed between a previous day and a present day during the cell culture process when the residual amount of glucose is greater than the predetermined glucose target;
determining the consumed amount of glucose based on a difference between the predetermined glucose target and the residual amount of glucose when the residual amount of glucose is not greater than the predetermined glucose target;
calculating an integrated viable cell density;
calculating a predetermined viable cell density for a following day based on the integrated viable cell density;
calculating a specific glucose consumption rate based on the consumed amount of glucose and the integrated viable cell density;
calculating a predicted glucose consumption amount by multiplying the specific glucose consumption rate by the predetermined viable cell density for the following day;
calculating a glucose target by summing the predetermined glucose consumption amount and a predetermined glucose minimum amount; and
feeding glucose to the bioreactor according to the glucose target.

18. The method of claim 17, wherein the feeding takes place on a daily basis.

19. A system of controlling a nutrient feed in a cell culture process, comprising:
a processor in communication with a bioreactor comprising a cell culture and a nutrient feed system that feeds a nutrient to the bioreactor, the processor configured to:
determine a viable cell density and a residual nutrient measurement from a sample retrieved from the bioreactor;
calculate a daily nutrient feeding target based on the viable cell density and the residual nutrient measurement; and
direct the nutrient feed system to feed the nutrient to the bioreactor according to the calculated daily nutrient feeding target.

20. The system of claim 19, wherein the nutrient feed system provides a continuous or discontinuous feed of the nutrient during the cell culture process.

21. The system of claim 19 or claim 20, wherein the nutrient is selected from glucose, glutamate, galactose, lactate, and glutamine.

22. The system of any one of claims 19 to 21, wherein the nutrient includes one or more monosaccharides.

23. A system of balancing a glucose feed in a cell growth process, comprising:
a processor in communication with a glucose feed system that feeds glucose during the cell growth process, the processor configured to:
periodically determine a viable cell density and a glucose concentration measured during the cell growth process;
periodically adjust a glucose feeding target based on the viable cell density and the glucose concentration; and
periodically direct the glucose feed system to feed glucose to the cell growth process according to the glucose feeding target.

24. A system of preventing glycation in a cell culture process, comprising:
a processor in communication with a bioreactor comprising a cell culture and a nutrient feed system that feeds a nutrient to the bioreactor, the processor configured to:
determine a residual amount of a nutrient within a sample retrieved from the bioreactor;
determine a consumed amount of the nutrient since previous feeding based on the residual amount of the nutrient;
determine a viable cell density within the sample;
calculate a predicted consumption amount of the nutrient to be consumed before next feeding based on the consumed amount of the nutrient and the viable cell density;
calculate a target amount of the nutrient for current feeding based on the predicted consumption amount of the nutrient and a predetermined residual nutrient target before next feeding; and
direct the nutrient feed system to feed the nutrient to the bioreactor according to the calculated target amount of the nutrient.

25. A method of modulating an amount of glycation of an agent in a cell culture process, comprising:
receiving a sample from a bioreactor comprising a cell culture;
measuring a residual amount of a nutrient from the received sample;
determining a consumed amount of the nutrient since previous feeding based on the residual amount of the nutrient;
determining a viable cell density from the received sample;
calculating a predicted consumption amount of the nutrient to be consumed before next feeding based on the consumed amount of the nutrient and the viable cell density;
calculating a target amount of the nutrient for current feeding based on the predicted consumption amount of the nutrient and a predetermined residual nutrient target before next feeding; and
feeding the nutrient to the bioreactor according to the calculated target amount of the nutrient.

26. The method of claim 25, further comprising:
determining a predicted viable cell density between the current feeding and the next feeding based at least in part on the determined viable cell density;
determining a nutrient consumption rate based at least in part on the consumed amount of the nutrient; and
calculating the predicted consumption rate based on the predicted viable cell density and the nutrient consumption rate.

27. The method of claim 25, wherein the feeding takes place on a daily basis.

28. The method of any one of claims 25 to 27, wherein the nutrient is selected from glucose, glutamate, galactose, lactate, and glutamine.

29. The method of any one of claims 25 to 27, wherein the nutrient includes one or more monosaccharides.

30. A method of controlling a glucose feed in a cell culture process, comprising:
determining a glucose measurement;
determining a lactate measurement;
determining a current culture day; and
determining a glucose target based on a combination of the glucose measurement, the lactate measurement and the current culture day.

31. The method of claim 30, wherein the glucose target is 5 g/L, when the glucose measurement is less than 1 g/L, the lactate measurement is less than 1 g/L, and the current culture day is day 5.

32. The method of claim 30, wherein the glucose target is 4.5 g/L when the glucose measurement is less than 1 g/L, the lactate measurement is greater than 1 g/L and less than 3 g/L, and the current culture day is day 5.

33. A method of controlling a glucose feed in a cell culture process, comprising:
receiving a sample from a bioreactor comprising a cell culture;
measuring a residual amount of glucose from the received sample;
comparing the residual amount of glucose with a predetermined glucose target;
calculating a consumed amount of glucose by:
determining the consumed amount of glucose by determining an amount of glucose that is consumed between a previous day and a present day during the cell culture process when the residual amount of glucose is greater than the predetermined glucose target;
determining the consumed amount of glucose based on a difference between the predetermined glucose target and the residual amount of glucose when the residual amount of glucose is not greater than the predetermined glucose target;
determining a viable cell density of the present day;
determining a viable cell density of the previous day;
estimating a growth rate based on the viable cell density of the present day and the viable cell density of the previous day;
predicting an integrated viable cell density for a following day based on the estimated growth rate;
calculating a predetermined viable cell density for the following day based on the integrated viable cell density;
calculating a specific glucose consumption rate based on the consumed amount of glucose and the integrated viable cell density;
calculating a predicted glucose consumption amount by multiplying the specific glucose consumption rate by the predetermined viable cell density for the following day;
calculating a glucose target by summing the predetermined glucose consumption amount and a predetermined glucose minimum amount; and
feeding glucose to the bioreactor according to the glucose target.
